(19) United States
(12) Patent Application Publication
(10) Pub. No.: US 2002/0059030 A1
(43) Pub. Date: May 16, 2002

(54) Method and apparatus for the processing of remotely collected electronic information characterizing properties of biological entities

(76) Inventors: Michael J. Otworth, Melbourne Beach, FL (US); John S. Scott, Sebastian, FL (US); Enoch Scott Blackwell, Melbourne, FL (US); John A. McMorris III, Indialantic, FL (US); Meagan J. Packard, New York, NY (US); Andrew Cole, Palm Bay, FL (US); Gregory A. Mullins, Melbourne, FL (US); Galo F. Acosta, Sebastian, FL (US); Richard H. Ferrans, Austin, TX (US)

Correspondence Address: FINNEGAN, HENDERSON, FARABOW,, GARRETT & DUNNER, L.L.P., 1300 I Street, N.W., Wahington, DC, 20005-3315, US

(21) Appl. No.: 09/906,005

(22) Filed: Jul. 17, 2001

Related U.S. Application Data
(60) Provisional application No. 60/218,583, filed on Jul. 17, 2000.
(60) Provisional application No. 60/218,584, filed on Jul. 17, 2000.
(60) Provisional application No. 60/218,585, filed on Jul. 17, 2000.

(51) Int. Cl. G01N 33/48

(52) U.S. Cl. 702/19

(57) ABSTRACT

A method and system 1 for processing electronic information associated with pre-determined characteristics of a biological subject 10. A testing kit 100 is provided at a location proximate the subject 10 for obtaining a representative sample 115 of the biological subject 10. The testing kit 100 includes a cartridge 110 which performs a pre-selected test or series of tests and generates electronic information 70 representative of sample characteristics. The electronic information 70 is transmitted to a remote data service 200 to determine pre-selected test results. The data service generates and transmits the test results to a testing kit operator, the subject, a provider, or a pre-selected third party, as may be appropriate and also performs a wide range of attendant administrative functions.

A claim to priority is made to Provisional Applications Serial No. 60/218,583 for “Method And Apparatus For The Collection And Analysis Of Diagnostic Data;” Serial No. 60/218,584 for “Method And Apparatus For The Collection And Analysis Of Diagnostic Data—System Architecture;” and Serial No. 60/218,585 for “Method And Apparatus For The Collection And Analysis Of Diagnostic Data—Functional Requirements;” all having a filing date of Jul. 17, 2000, all commonly owned with the instant application, and all herein incorporated by reference.

### FIELD OF THE INVENTION

The present invention relates to the field of performing characterization testing on various biological samples and systems and to the information management of the resulting characterization data. These biological samples and systems may include human as well as animal subjects, botanical and agricultural subjects, and ecological and environmental systems. More particularly, the present invention, in various specific embodiments, involves methods, systems and apparatus directed to providing remotely collected information to a centralized testing service and, there, processing, managing and distributing that data and the associated processed data thereby enabling various applications and uses of that data.

### BACKGROUND OF THE INVENTION

Recently, there has been considerable interest in the development of smart sensors capable of discriminating different analytes, toxins, viruses, fungi, cells (including blood cells) and bacteria for a wide range of applications, including human and veterinary medicine, environmental, health and safety, remote sensing, food/beverage, agriculture and chemical processing. Sensors have been developed that detect a single analyte or multiple analytes. Sensors for multiple analyte detection generally include an array of sensors. The advantages of these array sensor systems are their ability to quickly analyze multiple analytes, and their ability to be “trained” to respond to new stimuli through the use of specific receptor molecules and the ongoing development of new types of molecules. The on-site adaptive analysis capabilities afforded by array-based sensors displaying the capacity to sense and identify complex gases and liquids have been fashioned using a number of distinct transduction schemes.

A number of different types of array sensors have been described. These include sensors generally referred to as “electronic noses” if the sensor samples air, as opposed to a liquid. Typical electronic noses fall into several categories including conductivity sensors, piezoelectric sensors, metal oxide silicon field effect transistor (MOSFET) sensing devices, optical sensors and spectrometry-based sensing methods.

In many applications, it is necessary to identify and quantify analytes present in either liquid or solid-phase samples. In these applications, the sensor is sometimes referred to as an “electronic tongue.” An electronic tongue is an instrument comprised of an array of electrochemical sensors with partial specificity and an appropriate pattern recognition system, capable of recognizing simple or complex fluids. A number of technologies can be used to construct individual sensors, each chosen for a particular sensitivity and environmental characteristic. Types of electronic tongue sensors include fiber optic sensors; those based on “DNA on a chip” technologies; thin films, dyes or beads that act as receptor units; and pulse spectroscopy cells. In all cases, fluid, gas or liquid is introduced to the individual sensors, either by passive exposure or by pumping the fluid sample across the individual elements, to induce an electrochemical reaction.

There are several areas where such technology in general, and the present invention in particular, can play an important role. Public awareness relating to food safety issues has increased significantly in recent days due to deaths related to E. Coli 0157:H7 deaths, as well as media coverage of BSE and FMD issues in Europe. The public wants to know if the food we eat is safe. The consumer expects zero “0” risk in the safety of their food, while retailers want to assure their customers of the safety of their food products as well as complying with government regulations. This is a domestic as well as an import/export problem further complicated by the increasing globalization of the food supply that requires countries to develop a global standard around food safety and concerns about food-borne pathogens and the use of antibiotics and their relationship to anti-microbial resistance.

Nucleic acid amplification, such as PCR (Polymerase Chain Reaction) or TMA (Transcription Mediated Amplification) provides a test that has the same consistent performance characteristics. When an agent such as E. Coli 0157:H7 infects a food animal, amplification works by chemically replicating a segment (target amplicon) of the E. coli nucleic acid that is unique to that species and/or strain. The replication takes place on the order of a billion fold, and detection is performed via a chemical marker. In one embodiment of the present invention, a plot of initial amplicon copies vs. replicated amplicon after amplification is used to quantify the infection in the animal or test material. In another embodiment, the target amplicon is present in the sample in sufficient quantity to be directly detected. In this case amplification is not necessary, and the system includes a direct detection method.

The “gold standard” of food testing used today is based on cell culturing. While a relatively accurate method of verifying suspected food contamination, typical cell culturing methods take between 24 hrs to 6 or 7 days to complete, meaning that the contaminant may not be detected until after the food has been consumed. One objective of the present invention is to replace the gold standard with a process that resolves the problems listed above for handling food safety.

In the areas of point-of-test and point-of-care medical care, the present invention can be helpful in many situations. For example, remote testing helps the medical environment by making office visits more efficient and productive. In the past, a subject was screened or tested under various protocols only after the subject physically visited a physician's office, a hospital, or a clinical laboratory environment. This often unduly inconvenienced the subject or the subject's family by undesirably imposing on their schedule, and also detracts from the efficiency of the care-provider's schedule. Moreover, patient visits to a hospital often involve exposing those in a fragile health-state to undue risk.

To avoid such inconveniences, the use of several types of remote test devices is commonly known. For example, a remote test kit for testing the glucose level in a subject's blood is known. One such example is a common home blood gluco-meter. Typically, this device requires a small drop of blood to be applied to a disposable cartridge that is inserted into a reading device. The output generally includes a glucose level value or, in the case of interference from unknown drugs, contaminants or out-of-date reagents, an error indication. By contrast, multi-analyte arrays intended for use in similar diagnostic scenario, such as those contemplated for use with the present invention, are able to monitor for these interfering conditions and correct the output using known correlation and calibration parameters. This may include multi-diagnostic panels or cartridges monitoring for ketoacids, fructosamine, renal conditions, or other common co-morbid conditions. In addition, these multi-analyte cartridges are capable of monitoring and correcting digitally for diminished potency reagents, enzymes, and signaling compounds.

Nonetheless, these and other such test kits are often available to a patient only after consultation with a doctor. Similarly, basic diagnostic tools currently available to patients, such as thermometers or blood pressure cuffs, facilitate only the most basic form of pre-visit screening and fail to automatically communicate test data back to the health care provider.

For many more complicated diagnostic tests, a subject must either visit a doctor or hospital, or receive a visiting medical professional at the subject's location, in order to submit to the test. Thus, advanced diagnostic tests for pre-screening of subjects have not been available in a form that allows a subject to be pre-screened without the intervention of medical or care providers.

There are problems associated with the billing aspects of remote testing. For instance, the authorization by some third party payers for the payment of testing costs may depend upon the individual subject and their particular situation and may only encompass the approval of one form of kit, while still another other third party payer, for the same subject or the same situation, may authorize yet a different kit—or no kit at all. Furthermore, when using existing test kits to remotely test for blood sugar levels, the purchaser of such test kits is known to have to arrange and purchase testing supplies that may be billed in one transaction, while the testing analysis may be billed in a second transaction. In other words, the potential for simplified remote testing has been hampered because the testing operator or subject has to deal with those third parties who are attempting to arrange payment for their segment of the total test expenses. There may be separate costs for the testing device, additional costs for the testing supplies, and even more costs to other parties, such as independent laboratories, for performing the test. Indeed, there may yet still be more costs and complex billing arrangements that must be setup with medical providers who would interpret the test results. Thus, the complexity of this kind of arrangement, which is common in conventional medical lab testing, often renders the use of remote testing devices unduly burdensome.

Furthermore, since these multiple transactions are further complicated by layers of insurance and often confusing billing procedures, each diagnostic test poses an undue burden on the billing system. Moreover, such multiple transactions can cause difficulties in bill tracking, both from the perspective of the medical provider and the subject. Therefore, there is a need for a system that avoids these problems when using remote testing devices.

Another aspect of remote testing which is addressed by the present invention is the frequent requirement for conducting the remote testing to predetermined schedules or regimes of testing wherein a subject must partake in a plurality of tests. For example, a medical patient engaged in a regime of blood sugar testing may be required to complete blood sugar tests at regular intervals, say once every other day. However, because the impetus for such testing rests solely on the subject, the subject may easily forget tests in a testing schedule. Further, the tests may be scheduled so far apart as to become easy to forget, as well. Moreover, a subject may completely cease participation in a testing schedule due to apathy, forgetfulness or laziness. As well, similar problems often exist regarding prescription compliance and its ultimate affect on the therapeutic disposition of a patient. There is a well recognized problem relating to subjects whose prescription regimen is based upon frequent testing and/or confirmation of their medical condition. In those circumstances where the subject is required to adhere to such a complicated or recurring test schedule, these tests are often performed either erratically or not at all, with the result that the prescribed treatment becomes inappropriate to address the underlying medical condition(s). This ultimately has a negative affect on the therapeutic disposition of the subject.

Known methods for carrying out a schedule of remote tests, while convenient in many other respects, lack the rigors of scheduled tests performed with a testing professional at a central office. Without such rigidity in a schedule of tests, the great benefits of remote testing are often obviated by a subject's inevitable failure to scrupulously complete the regimen of tests.

The remote testing associated with the present invention can also be helpful in many environments, including the medical field, because tests may be performed away from the diagnostic or data analysis center. For example, a blood sugar test may be performed away from a doctor's office or a hospital. Nonetheless, known methods of remote testing typically provide raw test data and results without the benefit of in-depth comparison of the test data results and statistical normalization with available repositories of previously collected data. For instance, the interpretation of test data and results may be greatly aided by correlating these data and results with known data. Without such comparison or interpretation or validation, the test results provide merely a cursory level of help for the subject being tested. The potential of further insuring the quality of the collected data and the associated processing is lost without the ability to perform this comparison. Moreover, current systems often lack facility for providing feedback regarding the proper operation of the test device or other related status information either to the patient or healthcare provider.

Unfortunately, known remote testing systems are typically limited by size and portability constraints. Thus, it is highly inconvenient and impracticable to tote large amounts of data, such as secondary databases in electronic format, in conjunction with a remote testing device. Even if the size and portability constraints were overcome, the inherent difficulty in connecting with these portable databases effectively puts their completeness and currency in serious question.

Yet another area in the medical field benefiting from the present invention relates to the screening of potential participants for a clinical trial or study. The existing methods for accomplishing this have not typically utilized a remote testing scheme with central data management. For example, potential human participants are often broadly solicited through public advertisements or notices. Unfortunately, such public announcements or notices typically do not lend themselves to a great deal of targeting toward desired segments of a general population. Typical solicitations are made, for example, on Internet sites, on radio programs, in newspapers, in magazines, and on television. Despite attempts to focus advertisements or notices in publications that target specific audiences, such attempts can still be characterized as low yield. Thus, such solicitations often waste valuable resources in an attempt to gather a limited and specialized segment of a population.

Moreover, testing of potential participants for inclusion in the clinical trial or study usually is conducted at a central office or laboratory, wherein a subject must travel, often a great distance, to the office/laboratory to be considered for the clinical trial or study. Such inconvenience will often lead to a smaller pool of potential participants, due to the apathy of subjects or an inability or reluctance of subjects to travel to the central office or laboratory.

Furthermore, a new process for screening potential participants must typically be devised for each new trial; such processes typically cannot reuse the screening mechanisms of past clinical trials or studies, due to dissimilarities among potential participants. Thus, new solicitations and processes must be implemented for each new clinical trial or study, which undesirably costs more money for each new clinical trial or study when no ongoing screening apparatus generally exists. As well, volunteers cannot be recruited in real-time, adding latency to the trial schedule. Therefore, there is a need for an improved system and method for screening a subject as a possible participant in a study or clinical trial that is more efficient, better yielding, and avoids the existing problems noted above.

Another area to benefit from the present invention is related to the scenario where a subject is placed in quarantine or an isolated situation pending some determination of whether the subject is a threat. Typically, a subject may be isolated or restricted of free movement to prevent the spread of a health risk, such as a contagious disease. Such isolation may include a quarantine of a human, agricultural, or animal subject due to a suspected or actual disease, a quarantine of an immigrating subject based on immigration laws, or isolating a source of drinking water or isolating a body of water from human contact.

Typically, when a subject is isolated or quarantined, it is desirable to test underlying facts or reasons for the isolation or quarantine. In this way, the reason for the isolation or quarantine may be obviated or justified. If a subject no longer exhibits the reasons for the quarantine, a clearance may be granted. If the reasons for the quarantine persist, the quarantine may be continued.

However, testing the reasons or underlying facts for quarantine can be a time-consuming and inconvenient process. For example, when a test requires the acquisition of a sample and secondary processing at a remote laboratory—thus taking a substantial time period—the quarantine or isolation may be unnecessarily prolonged. Further, an isolated test subject or a test subject related to a quarantine, such as a body of water or a bedridden patient, may not be moveable to a central testing location for expedited testing. In a similar manner, central testing locations may deny access to quarantined subjects for fear of the potential of further spreading of contagious diseases that initially gave rise to the quarantine. Likewise, quarantined subjects may not be able to travel to central testing a location, since the quarantine may, by its very nature, restrict the movement of the subject. Therefore, there is a need for improved methods and systems for testing or screening isolated or quarantined test subjects that advantageously use remote testing devices.

While most of this background information describes the human medical field of use, there are similar areas of interest in the fields of animal medicine and environmental/ecological systems where the present invention brings great benefit. These are discussed later in their specific related embodiments.

In short, there exist several areas of concern in human and animal medicine as well as agricultural and environmental systems for which the present invention provides an innovative solution. The new and useful uses and configurations for a centralized and consolidated data collection and processing service as described herein functionally integrated known and future sensor-based systems at various remote locations and further consolidate and integrate the resulting data to address these known areas of concern.

### SUMMARY OF THE INVENTION

In accordance with the invention, in view of the foregoing background, it is therefore an object of the present invention to provide an integrated system in which biological sample characterization may be achieved using one or more sample data collection devices, or sensors, in conjunction with a centralized remote data analyzer. For the purposes of this application, a biological sample is considered to be any portion of a subject or system, either living or dead, where the subject or system includes human, animal, botanical or agricultural and ecological or environmental. Furthermore, the biological sample may be in the form of a gas, a liquid, a solid or any combination thereof.

These applications and uses are described in greater detail within this specification and, by way of example, include: helping to ensure food safety, allowing effective and convenient pre-screening of patients prior to scheduling office visits, enabling pharmaceutical development by streamlining pre-clinical and clinical testing, simplifying the reimbursement of the costs associated with diagnostic testing, and more efficiently monitoring and controlling inventories of products used in the conduct of diagnostic testing.

The present invention can also be understood as a system for collecting, managing, normalizing, and correlating the collected analytical data and the resulting processed data at a location remote from the points of collection of the biological samples. By definition, the point of collection of the sample must be proximate to the subject or system of interest and is often referred to as the “point of care.” Thus, the present invention provides a system for sample characterization at the point of care.

The method and system of the present invention are based on the use of a sensor capable of translating chemical or physical properties of a collected sample into electronic information or data. An example of such a sensor is one comprised of a substrate having a plurality of spatially distinct locations, wherein at least one of the spatially distinct locations is associated with a reactant. The substrate is any material capable of having a reactant incorporated therewith. A reactant may optionally be associated with a support member. In particular, the reactant may be attached to the support member, or may be intercalated or otherwise incorporated within the support member. The spatially distinct locations are comprised of locations spaced apart from one another on the substrate. These locations may be in an ordered array or may be unordered. Typically, the substrate defines a plurality of cavities, with at least one cavity representing at least one of the spatially distinct locations. The reactant is any material capable of interacting with an analyte such that changes occur in some characteristic(s) that are detectable and that are indicative of the analyte present. The reactant may be a sensing particle, a receptor molecule attached to a sensing particle, a gel, or a reactive coating.

Although any number of types of sensors may be utilized for the characterization of the sample of interest, a preferred sensor structure is based on utilizing a sensor array containing a plurality of sensing particles, which preferably include receptor molecules. In particular, after the receptor molecules interact with the analyte of interest in the sample of interest, the receptor molecules experience spectroscopic changes that may be detected using a suitable detector. This preferred sensor structure is discussed in detail in the above noted U.S. Applications.

A key element of the present invention is the centralized remote data analyzer that links individual sensors. This centralized remote data analyzer, or data service, is electronically connected to the sensors through any one or more of known networking techniques including wireless, internet, intranet, modem, ethernet, etc. It can be comprised of one or more computers configured to communicate with the sensors over the selected communications network and to receive the electronic information transmitted by the sensors. The data service can also be configured to perform one of several additional tasks including storing the received information such that it can be retrieved at a later time, processing the received data to determine the sample characterization results associated with the received information, storing the sample characterization results, transmitting the sample characterization results to one or more interested parties, performing correlation analyses of the sample characterization results with existing databases of related information and results, correlating clinic to clinic or even chip to chip results, data mining, including looking for suspected relationships between fields of data and using general data visualization techniques to discover heretofore undiscovered correspondences between data. In many regards, the system can also be configured to be “self policing”, by maintaining secure audit trails (maintaining records of data accesses, credentialing, authentication, etc.), by performing inventory management (tracking facility or user test kit use; maintaining test kit expiry data, shelf-life data, etc.) and so forth.

The present invention further expands the methods and uses of the systems described in application for patent U.S. Ser. No. 09/571,304, entitled Method and System for Remotely Collecting and Evaluating Chemical/Biochemical Information, filed May 8, 2000, and provisional application for patent U.S. Appl. No. 60/202,839, entitled Methods of Using a Sensor Array for Biochemical and Chemical Analysis, also filed May 8, 2000, both of which are incorporated herein by reference in their entirety.

When configured for a food safety application, the present invention allows the remote collection of sample information where the selected sample or samples are inserted into a sample preparation system. If appropriate this system first pre-processes the sample chemically (adding reagents) or physically (e.g. through liquification, heating, etc.). A portion of the prepared sample is further processed to isolate a target component from the rest of the sample. The nucleic acid extract or the remaining solution that contains the target component is then inserted (manually or automatically) into a processing station. This processing station amplifies or isolates the target component, depending on the desired characterization. The processed data is then electronically transferred into a data manipulation system that processes the data and transmits the results to the test site or other databases.

Such an architecture allowed by the present invention enables the analysis of the collected sample characterization information to be separate and distinct from any sample gathering and/or sensing. This approach is quite different from traditional “wet lab” analytical procedures in which the sample itself is physically handled at the analysis site and studied by instrumental or chemical means. According to the present invention, the sensing is complete at the moment the reagent-containing cartridge interacts with the sample and only an appropriate form of electronic data is transmitted to the remote analytical location.

Diagnostics according to the present invention are achieved without the need to physically transport, handle or manipulate a sample at any location separate from that of the sample origin such as a laboratory, for example, as is typically the case for conventional analytical and diagnostic testing. The present invention represents the first system in which analysis may be achieved at a location remote from the point of sample collection and/or handling, thus enabling diagnosis at the point of care, while maintaining statistical controls. Further, the present invention provides the first instance of an integrated system wherein sample diagnostics are achieved without the need for the physical presence of the sample at the analytical site. The present invention also provides for rapid availability of results in comparison to the current state of the art. While “state of the art” varies by application, results are typically available from the present invention in minutes to a few hours. In meat processing, for example, having results available within 4 hours is tremendously enabling in comparison to the currently-accepted industry norm of 72 hours, which potentially allows the questionable food products to already be consumed.

The present invention provides a novel and unique method of pre-screening a subject, particularly for human medicine applications, prior to committing to or arranging for bringing the subject and the test provider together. In this application from a care provider perspective, the present invention is configured to include determining a set of sample characterizations, or “test”, to be administered to the subject. The test allows for the detection of some number of specific constituents contained within a sample of bodily fluid obtained from the subject. The test for each of these specific constituents is considered to be an “assay” and these multiple assays may be physically housed within a portable “cartridge”. In one embodiment a kit is provided for the subject. The kit includes a selected cartridge or a set of multiple cartridges that is related to a desired test. Instructions are also provided to inform the subject or a test operator of when and how to collect and administer the required fluids to the cartridge and how to introduce the cartridge to the remote testing device such that the individual assays may be examined and the resulting information transmitted. Electronic data related to the pre-determined test and local test conditions is received from the remote testing device at a central data facility after the remote testing device conducts the determined test using the selected cartridge or set of cartridges. The electronic test data is processed at the data service in order to determine test results. The test results are then transmitted from the central data service to one or more third parties.

In another aspect of the present invention, a method is described for pre-screening a subject using a remote testing device from the perspective of a subject. This alternate method comprises receiving a kit having the remote testing device and a disposable cartridge or set of cartridges and associated instructions for how to administer the test or tests. The disposable cartridge or set of cartridges contains the reactive elements to support the test or tests that have been preselected by a third party care-provider. The subject or test operator follows the instructions for providing a sample or samples to the identified sample input port(s) on the cartridge(s) and each cartridge is inserted in-turn into the remote testing device where the changes to the reactive elements are observed and representative electronic test data is generated. This electronic test data is transmitted to a central data service prior to a visit with the third party care-provider.

The present invention also enables the simplification of the billing aspects associated with remote testing. In one aspect as broadly described herein, one embodiment of the present invention includes a method for prepaid testing using a remote testing device. The method comprises preparing a kit having a replaceable cartridge associated with one or more tests and an appropriate reader. A prepaid analysis fee is incorporated into a price for the kit. The kit may then be sold or given to a subject. Generally, the prepaid analysis fee comprises a first fee for centrally analyzing electronic test data transmitted from the remote testing device and a second fee for providing test results to an appropriate recipient.

In another aspect, another method for prepaid testing using a remote testing device is disclosed. The method comprises preparing a customized kit for sale to a predetermined type of purchaser, wherein the customized kit comprises a replaceable cartridge or set of cartridges associated with a test. The customized kit is priced to include a prepaid analysis fee into a price for the customized kit. The prepaid analysis fee represents a first fee for accessing a central data facility after the remote testing device conducts the determined test using the replaceable cartridge or set of cartridges from the customized kit. The prepaid analysis fee further represents costs involved with analyzing electronic test data transmitted from the remote testing device to the central data service and storing the test results.

The present invention also provides new and useful configurations and uses for a sensor-based remote testing system and method for centrally correlating remote testing data. In one aspect as broadly described herein, a method is disclosed for remote testing a sample from a subject using centralized correlation. The method comprises collecting electronic data on the sample with a remote testing device. Electronic data are sent to a central data service and the electronic data are analyzed by the central data service to provide test results. The test results are then advantageously correlated with a secondary data set by the central data service to yield relationship information associated with the test results. The relationship information may include one or more similarities with the secondary data and may also include trending information. This comparison information enables a statistical analysis of test results and improvement of the quality of the data generated thereby leading to improved patient management. The secondary data set may comprise historical data about the subject, treatment data, or side effect data.

In another aspect, a subset or particular elements of the test results are compared to another subset or elements of the test results as the secondary data set. One or more similarities are identified from the comparison. These similarities include relationship information about the test results, such as trending information. From the test results, conclusions may also be drawn, such as a medical diagnosis or an identification of the source of a particular pollutant in a body of water.

The present invention provides new and useful configurations and uses for remote testing a subject according to a centrally managed schedule of tests. In one aspect as broadly described herein, a method is disclosed for remote testing a subject according to a centrally managed schedule of tests. The method comprises establishing the centrally managed schedule of tests at a data service based upon historical data related to the subject. An appropriate testing kit is then provided the subject, the testing kit including at least one replaceable cartridge and possibly a cartridge reader. The replaceable cartridge is related to one of the tests determined in the schedule. A reminder is received from a central data service before one of the tests. Electronic data are collected on a sample associated with the subject using a testing device and the electronic data are sent to a central data service. The electronic data are analyzed according to one of the tests in the centrally managed schedule of tests. Test results from the analysis of the electronic data are stored on the central data service. An entry associated with the one of the tests is highlighted, wherein the entry is associated with the centrally managed schedule of tests. A determination is made if the centrally managed schedule of tests is complete, and a reminder is issued if the schedule is not complete.

The present invention also provides new and useful configurations and uses for a sensor-based remote testing system and method for screening a subject as a potential participant in a study, such as a clinical trial. In one aspect as broadly described herein, a method is disclosed for screening a subject as a potential participant in a clinical trial using a remote testing device. The method comprises receiving electronic test data at a central data service from the remote testing device, wherein the electronic test data is related to a sample from the subject. The electronic test data are analyzed to produce test results. The test results are then centrally compared to at least one requirement for the clinical trial, in order to screen the subject as the potential participant for the clinical trial.

Optionally, upon the conduct of the data comparison and if the test results meet the at least one requirement, a message is sent to a third party, such as a clinical trial test manager or the laboratory director of the healthcare facility wherein the subject may be tested. The message may indicate the existence of the potential participant for the clinical trial. The third party may be billed for an initial amount if the test results meet one or more of the requirements for the trial. At the discretion of the subject's healthcare provider or, more generally, the test operator, the subject may be notified regarding being the potential participant for the clinical trial. At the discretion of the appropriate healthcare provider designee, such as the subject's doctor or the facility's laboratory director, or of the subject themselves, an authorization message may be received by the clinical trial manager or chemical monitor of the sponsor company from the subject related to the clinical trial. In this manner, the third party may be notified regarding the subject's authorization message and be billed for an additional amount. This also provides the subject the opportunity to determine the degree of their personal information to be associated with any of the data resulting from or generated for the trial or testing in question. The subject's healthcare provider may optionally receive a fee for facilitating the identification and/or enrollment of a clinical trial candidate.

The present invention also provides new and useful configurations and uses for screening a quarantine subject using a remote testing device. In one aspect as broadly described herein, a method is described for screening a quarantine subject using a remote testing device. The method comprises gathering electronic test data on a sample at a quarantine facility, wherein the sample is related to the quarantine subject. The electronic test data are sent to a central data service for analysis. A granting notification is received from the central data service based upon the analysis of the electronic test data. A clearance may be granted to the quarantine subject based on the granting notification.

It is to be understood that both the foregoing general description and the following detailed description are exemplary and explanatory only and are not restrictive of the invention, as claimed.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate one (several) embodiment(s) of the invention and together with the description, serve to explain the principles of the invention.

### BRIEF DESCRIPTION OF THE DRAWINGS

A preferred embodiment of the invention and alternate embodiments are described, by way of example, with reference to the accompanying drawings, in which:

FIG. 1 is a functional block diagram of an embodiment of a testing kit for characterizing samples obtained from a subject consistent with an embodiment of the present invention;

FIG. 2 is a functional block diagram of an exemplary system environment for characterizing samples obtained from a subject utilizing a remote testing kit consistent with an embodiment of the present invention;

FIG. 3 is a flow chart of an exemplary method for testing and data management services utilizing a remote testing kit consistent with an embodiment of the present invention;

FIG. 4 is a more detailed flow chart of an exemplary subroutine used in the method of FIG. 3 for collecting electronic data on samples with a remote testing device consistent with an embodiment of the present invention;

FIG. 5 is a more detailed flow chart of an exemplary subroutine used in the method of FIG. 3 for sending electronic data to a central data service consistent with an embodiment of the present invention;

FIG. 6 is a more detailed flow chart of an exemplary subroutine used in the method of FIG. 3 for analyzing electronic data by a central data service consistent with an embodiment of the present invention;

FIG. 7 is a flow chart of an exemplary method for pre-screening a subject, shown from the perspective of a testing provider, consistent with an embodiment of the present invention;

FIG. 8 is a flow chart of an exemplary method for pre-screening a subject, shown from the perspective of the subject, consistent with an embodiment of the present invention;

FIG. 9 is a flow chart of an exemplary method for remote testing a subject according to a centrally managed schedule of tests consistent with an embodiment of the present invention;

FIG. 10 is a flow chart of an exemplary method for remote testing incorporating a reminder function consistent with an embodiment of the present invention;

FIG. 11 is a flow chart of another exemplary method for completing a schedule of remote testing with a reminder function consistent with an embodiment of the present invention;

FIG. 12 is a flow chart of exemplary method for completing a schedule of remote testing, shown from the perspective of a test subject, consistent with an embodiment of the present invention;

FIG. 13 is a flow chart of an exemplary method for screening a subject as a potential participant for a clinical trial using a remote testing device consistent with an embodiment of the present invention;

FIG. 14 is a flow chart of an exemplary method of screening a subject for participation in a clinical trial, shown from the perspective of a subject/potential clinical trial participant, consistent with an embodiment of the present invention;

FIG. 15 is a flow chart of an exemplary method of billing related to screening a subject for participation in a clinical trial consistent with an embodiment of the present invention; and

FIG. 16 is a flow chart of an exemplary method for screening a quarantine or otherwise isolated subject using a remote testing device, shown from the perspective of a testing provider, consistent with an embodiment of the present invention.

### DESCRIPTION OF THE EMBODIMENTS

Reference will now be made in detail to the present embodiment(s) (exemplary embodiments) of the invention, an example(s) of which is (are) illustrated in the accompanying drawings. Wherever possible, the same reference numbers will be used throughout the drawings to refer to the same or like parts.

The present invention will now be described more fully hereinafter with reference to the accompanying drawings, in which preferred embodiments of the invention are shown. However, this invention may be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

As described above, the present invention may be described as a system for collecting, analyzing, storing, processing and correlating electronic information about certain aspects of a biological sample, that system comprising one or more data collection devices and a data service which are functionally and physically separate. Such a system has a wide variety of applications, each of which enables new and novel benefits to its specific field of use, depending upon embodiments of the present invention selected for implementation. It will be understood to those of skill in the art, however, that aspects of the present invention may also be used to provide physical testing of other types of data and management and collection of that data.

A remote testing kit system becomes most powerful when associated instrumentation may be delivered and utilized at the application site. That is, rather than remotely collecting the samples and transporting them to a centrally located analysis site, it may be advantageous to be able to conduct the analysis at the sample collection location. Such a system may be used, for example, for: point of care medical diagnosis; in emergency care settings; on site monitoring of process control applications; military intelligence gathering; environmental monitoring; food safety testing; and etc. According to one embodiment, a remote testing kit includes a cartridge and a cartridge reader, the later further including a light source, a sensor array and a detector. The cartridge reader has a size and weight that allows the device to be easily carried by a person to a testing site.

In this exemplary embodiment, the present invention can be understood as a system 1 for collecting and managing point of care medical data related to one or more properties of a biological sample of interest. In accordance with embodiments of the invention, a sample is generally obtained from a subject and is introduced to a testing kit. In the example of medical uses of the current invention, the sample may comprise a bodily substance, such as blood, urine, fecal matter, or saliva, for example. The testing kit then produces data relating to the sample and transmits the data to a functionally centralized data service. The centralized data service analyzes the data to provide results. The centralized data service may archive the data, transmit results, and bill a party for a fee related to the test.

Additional discussion is now presented to further describe potential testing kit configurations where, in the embodiments described here, are primarily comprised of at least one cartridge within which is housed some sort of assay implementation, and a physically separate cartridge reader. The following descriptions of the present invention again, use the human point of care medical application as an exemplary embodiment but identify alternative features or considerations necessitated by other applications as appropriate.

In this exemplary embodiment and in general terms, a sample is taken from a subject of interest, selected based upon the characteristics to be measured or identified and upon the associated requirements of the related testing kit to be used. The sample is typically a biological sample and can be comprised of a fluid, a gas, or a combination of both. The sample is then prepared, if required, including potential liquification, treatment with specific reagents, temperature adjustment, or similar chemical or physical property modifications. When properly prepared, the sample is introduced to a cartridge or a sample transport device which is then introduced to a testing kit. Introduction may be accomplished in a variety of settings, including in situ, in vitro, or in vivo, with respect to the subject or source of origin of the particular sample.

The ability of a remote testing kit to be used for a variety of different types of testing will depend on the nature of the cartridge or sample transport device used. One embodiment of a cartridge is based upon a sensor array which includes a plurality of chemically sensitive particles, each of the particles including receptors specific for the desired task. For example, a sensor array cartridge for use in medical testing for diabetes may include a number of particles that are sensitive to sugars. A sensor array for use in water testing, however, would include different particles, for example, particles specific for pH, and/or metal ions, and/or other species of interest.

Such sensor array cartridge-based systems are known. An embodiment of a portable system for a sensor array is depicted in the referenced U.S. Appl. Ser. No. 60/179,293 which is incorporated herein by reference in its entirety. Using this application as an example, the sensor array cartridge may be held in place in a manner analogous to a floppy disk of a computer as described previously. In one embodiment, fluid samples may be introduced into the system at ports at the top of the unit. An inlet port may be used for the introduction of liquids found in the environment and some bodily fluids (e.g., water, saliva, urine, whole blood, etc.). As will be understood by those knowledgeable in the field of diagnostic devices, any port may be adapted to accommodate different input configurations, such as: capillary tubes, syringes, Luer lock adapter and the like.

In one embodiment, all of the necessary fluids required for the chemical/biochemical analyses are contained within the portable sensor array system. Fluids may be stored in one or more removable cartridges. Thus, when a cartridge is emptied of fluid, the cartridge may be replaced by a new cartridge or removed and refilled with fluid. Thus, fluids may be customized for the specific test or tests being run. In one embodiment, reader display screen may be used to provide sensor array identification information, and/or information relevant to the chemistry/biochemistry of sample being tested.

In an alternate embodiment to the approach described above, the sensor array chip may be housed in a cartridge that permits sampling and testing to occur in a self-contained manner. That is, the cartridge is the portable device used to contain sensor chip/array and introduce fluid of interest to the sensor array. The sensor array is a static device. An individual using the sensor array (e.g. hospital technician, nurse, etc.) uses the cartridge containing the sensor array as a self-contained device. The cartridge has everything built into the device that is needed for testing. It is capable of containing whatever sample volume is necessary (on the order of nanoliters to tens of milliliters.) The cartridge is used with a reader as mentioned above and described more fully below, usually by inserting the cartridge into a cartridge reader. The cartridge reader is the device at which interconnectivity exists.

There are additional optional cartridge features enabled by the present invention. The cartridge may also incorporate an integral sample filter used to filter whole blood into blood plasma or serum or to eliminate unwanted components in the sample prior to the performance of the intended testing. Again, as in the case of medical point of care applications, such a filter may be used to separate out the particulates and/or large cells in a blood sample thereby leaving only the blood serum to undergo the testing.

Also, the cartridge may incorporate an optional door, shutter, flap or cover such as that used on a typical floppy diskette. If testing kit is based upon optical changes occurring within the cartridge to indicate the characteristics of interest, such coverings can be incorporated that are transparent so as not to restrict the cartridge reader from detecting the anticipated changes in the liquid and sensor array.

If the reactions within the cartridge require the application of sample, additional reagents, wash solutions, etc. that result in left-over material not needed in the active area of the cartridge, an optional “waste” reservoir may be integrated into the cartridge. For embodiments such as those in agriculture or environmental where larger amounts of sample may be available, the cartridge may utilize a flow-through design where these waste materials are handled by incorporating input and outlet ports into the cartridge. In not having to accommodate for the integral storage of these materials onboard the cartridge, this approach allows for greater flexibility in the design of the assay represented by that cartridge.

An exemplary embodiment of the cartridge reader will now be defined more fully, beginning with a general description of its functionality followed by a more detailed description of its possible configurations as well as more information regarding its functional components.

An embodiment that is based upon observable optical changes related to certain characteristics of a biological sample, uses a cartridge reader whose primary function is to translate optical changes that occur in a cartridge to electronic information and to transmit that information to a data service. The reader provides integral optical illumination of the cartridge, electronic sensing of the resulting optical signature and the communications interface to the data service.

The illumination provided by the reader is selected based upon the specific requirements of the assay(s) implemented in the cartridge and the associated optical properties and changes anticipated from the conduct of the testing. Certain assays are based upon fluorescence where the reactive sites are illuminated by one wavelength of light and the resulting reaction of the sample with the cartridge reagents cases the reactive site to emit light at a different wavelength. Still other assays are based upon changes spectral properties of the reflected or transmitted light while still others utilize chemilluminescence where the reaction of the sample with the cartridge reagents generates light of an expected wavelength directly.

At the appropriate time, the reader allows the reflected, transmitted or generated optical signature from the active sites of the cartridge to fall upon opto-electronic sensors that convert the received optical signature into associated electronic information. At this point, prior to transmission to the data service, the reader performs pre-processing of the electronic information, i.e. it parses the complete data set collected into the minimum amount of data needed to accurately describe the observed property changes. In one embodiment, electronic images of beads are processed to remove those portions of the images not containing the beads. In some instances, the pre-processor may also enable or perform auto-calibration or device normalization, or certain optional signal massaging or characterization. These features will ensure more rapid data transmission via whatever mechanism is ultimately chosen for data transfer from the reader to the remote data site. The pre-processor may also perform other helpful functions including, but limited to, the compression of the original optical signature information to “pre-classify” each active site as being nominally a specific color value; or etc.

The active sites within a cartridge may be imaged sequentially, i.e. by element, by line or by row, as a complete array, or—for that matter—even through the ultimate concatenation of smaller arrays (i.e., by “tiling” a picture together). The optimal technique for collecting the required optical information must be selected based on balanced system-level considerations of factors that include, but are not necessarily limited to: electronic performance, physical constraints, imager cost, total reader cost, as well as combinations of any of the foregoing, etc. Furthermore, to successfully collect the required optical information, it should also be noted that it is not necessary to use a color image sensor. Different imaging techniques may be used, for example, to compose a color image; for instance, a monochrome array with sequential viewing of the target illuminated with different colors, followed by a step that combines the individual color planes to compose an integrated color image.

Another characteristic of the cartridge reader may be to ascertain cartridge identity information. Such information may be available from the cartridge either through optical information located appropriately upon the cartridge to be sensed by the reader or through some form of electronic data storage such as a magnetic stripe, an integral EPROM, etc. The reader may either store this information or, if appropriate, transmit it to the data service, either interposed with the sample information, or separately.

The reader then transforms the collected electronic data into a form that is suitable for transmission over the network interconnecting it to the centralized data service, and into a protocol that is expected by the centralized data service. It then communicates the transformed electronic information to the data service as appropriate and, depending upon the protocol of the communications with the data service, may await further instructions.

The next main component of the present invention is an interface or communication module. This reader functional component converts an electrical signal from the pre-processor and performs the actual transmission of the collected electronic information to the data service. Some of the signal conversions which may be implemented by this component include: a computer signal for internet transmission (includes USB or other serial connector conventions; etc.); modem signal for transmission over land-line or wireless phone line, or cellular phone; radio frequency; microwave frequency; infrared (sometimes preferred in EMI-sensitive environments); and any other communication technology developed in the future.

The present invention includes provisions necessary to incorporate the handling of encryption, public key infrastructure (PKI) issues, certificate handling, etc., within the biometrics provided or as part of the transaction overhead. For instance, all communiques back and forth between the reader and the remote data analysis site relating to PKI issues include any considerations for digital certificates used by certification authorities such as encryption keys or other encryption methodologies. The present invention includes provisions to ensure that encryption keys are properly handled such that data is encrypted according to the key. This is an important feature of communicating securely between the reader and the remote diagnostic communication server.

An optional feature of the present invention is a user or operator interface. A user interface may have more than one configuration, depending on location of use, sophistication of user, etc. Instances where a user interface may be optional would include, the case where all information is previously coded into a cartridge. This may be found, for example, for home use situations, where the use of a chip is “prescribed” for a patient by a physician or other health care provider.

In hospital, clinic or remote base scenarios, an individual (e.g.: nurse, health care provider, trained aide, etc.) may enter patient information (e.g.: scan a patient's identification (ID) bracelet, patient's UPC code, uses patient's biometric ID, etc.) along with unique cartridge identity information. The system 1 of the present invention would also allow a nurse or other health care provider to also enter their own code or other unique identifier at this point. This would ultimately enable and provide a check on “secure auditing” that is often associated with federal regulatory schemes, such as those outlined by the Health Insurance Portability and Accountability Act of 1996 (HIPAA), etc.

The present invention optionally includes alternative means for incorporating additional electronic information relative to the subject, test operator, the testing kit or other pertinent descriptive data. These alternative means include the ability to read bar codes, 2D symbology codes, magnetic stripes or other data storage media that may be included in a patient's file or from cartridge inventory. Additionally, the present invention provides the ability to interface with a keyboard or other data entry devices to collect additional information that becomes part of the test record.

In embodiments of the present invention where the testing occurs away from a care provider's facility, such as at the patient's home, the testing kit operator (the patient or another user) enters information or directs an interface to reference already coded info at a website to identify the transmission locale or other pertinent personal information. Alternately, the reader may already have the patient's name (or operator's name), user ID, treating physician or health plan, etc., stored in its memory and may simply require biometric input (such as fingerprint, iris scan, PIN, facial scan, etc.) or other patient or user authentication information to authorize processing or transmission.

Further elaborating upon the authentication aspect of the present invention, the incorporation of a biometrics capability into a cartridge reader can enable the authentication and/or identification of the patient, health care provider, etc., or to associate their action(s) with the sample. This is beneficial for various matters related to: the physical sample and the cartridge, i.e., situations involving sample control, custody, tracking or actual handling; to review, establish or prove professional oversight; and to establish a secure audit transaction trail, etc.

Furthermore, a biometric reader or other biometric input device may be used at any point in the present invention in order to initiate a variety of functions. Such functions may include; the pre-processing of the original optical characterization information; the transmission of the original optical characterization information to the data service; the initiation of analyses of the raw data at a remote database; the initiation of a billing sequence or cost-charge function; the initiation of reporting of results from the data service to a doctor or other health care professional; and any combination of the foregoing. It is important to note that the present invention allows centralized processing such that, at this point, there has been no analysis performed related to the characterization of the original sample or a determination of an associated diagnosis, if in the case of a medical application.

Depending upon the assay implementations supported by the cartridge reader, its configuration may include a portable, hand-held configuration, or it may be slightly larger, not allowing it to be considered a true handheld device but may still be considered mobile. The reader may also require such diversified illumination capabilities or a capacity to accommodate diverse reagent introductions that it could be a fairly large and complex device that must be physically mounted to a fixed location, making it a stationary implementation.

In the later case, the sample of interest could be introduced to a cartridge at the subjects' location and the exposed cartridge then delivered to the cartridge reader location. Patients may come to the reader, as opposed to a nurse in a hospital being mobile and going to individual patients. In a similar example, the reader might be located in a drugstore/pharmacy and at-home patients can bring their cartridge to the reader to be read, interpreted and uploaded to centralized data service for interpretation and diagnosis. By the same token, if cartridges used in a non-healthcare setting, such as a home, are controlled by prescription, etc., they could be issued at the pharmacy, used then and there, and subsequently read/uploaded by the reader.

An example of a stationary configuration is an application where the reader is part of desktop or tabletop model, and intended to be used at a designated site. In this instance, the reader would stay primarily connected or attached to an interface/communications module. With stationary readers, a cartridge is brought to the reader. The size of a stationary reader may be larger than the truly portable version(s) and, because of this additional latitude, may incorporate additional features and capabilities. Patients come or cartridges are brought to the reader, as opposed to a nurse in the hospital being mobile with hand-held device and going to individual patients.

Another scenario for a stationary reader would be an application where such a device was located in a drugstore or pharmacy. At-home patients could bring their cartridge to the reader to be read, interpreted and uploaded. Alternately, cartridges that are controlled by prescription or for use in a non-healthcare setting could be used immediately when purchased at the pharmacy, and read or uploaded by a reader located at that drugstore/pharmacy.

An example of an application benefiting from mobility of the testing kit is as used in an ambulance, Red Cross vehicle, etc. Here, certain well-defined tests are anticipated thereby helping to narrowly define the necessary cartridges and assays to be performed. The reader may or may not be hand-held, but will in all probability utilize wireless communications. In one embodiment, an interface may exist with an Emergency Vehicle (EV) communications systems with wireless transmission. For example, Nextel is experimenting with “as needed” wide-band cellular links for emergency vehicles. In such instances, the need to relay information to hospitals would override other non-urgent communications, and readers would be enabled to dial a hospital automatically. This would free emergency personnel for other care matters en route.

Using this later example, an ambulance crew, during the transport of an emergency patient, has the choice of which hospital receives their patient information. If information is sent to the wrong hospital, one advantage of the modular system 1 of the present invention is that information can be immediately re-transmitted to next hospital/clinic, without needing to repeat any testing.

Reference will now be made to various embodiments according to this invention, examples of which are shown in the accompanying drawings and will be obvious from the description of the invention. In the drawings, the same reference numbers represent the same or similar elements in the different drawings whenever possible.

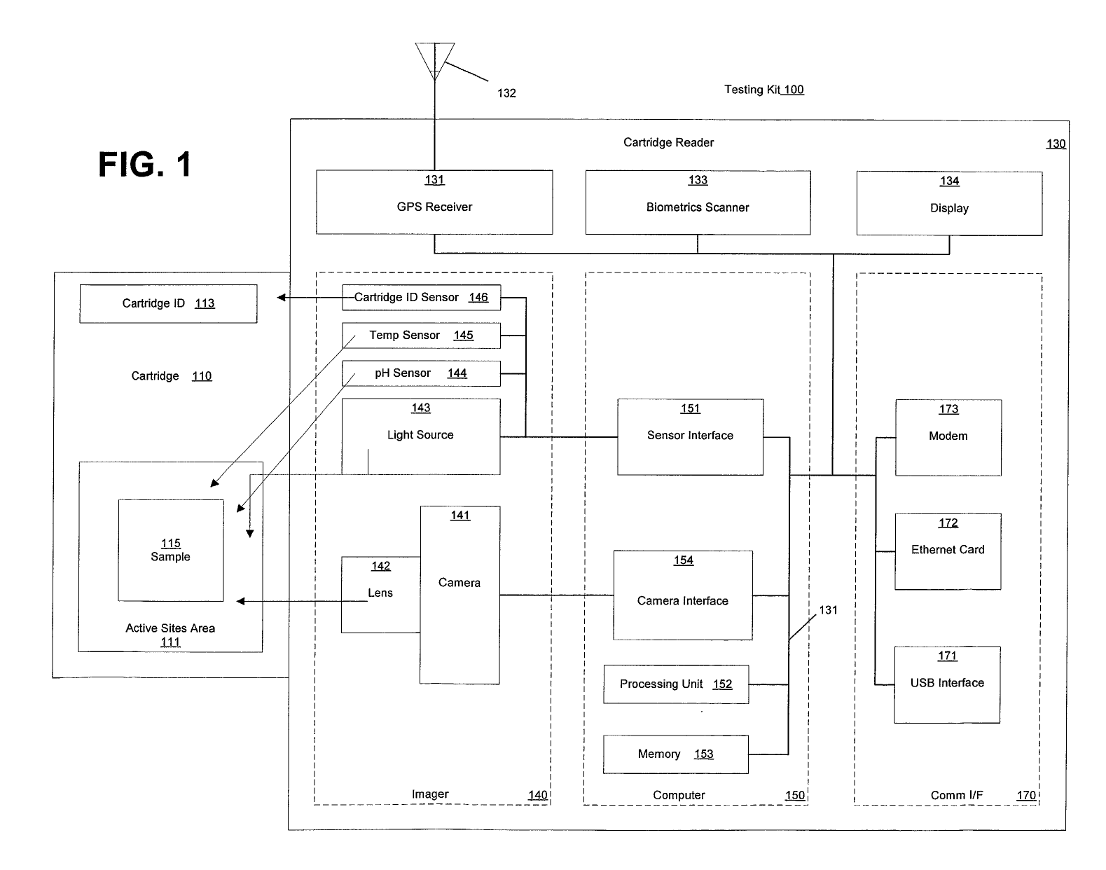

FIG. 1 illustrates an exemplary testing kit 100 in accordance with an embodiment of the present invention. The exemplary testing kit 100 comprises at least one or more selectable and separable cartridges 110 and may, in addition, include a cartridge reader 130. In turn, cartridge reader 130 comprises an imager 140, a computer 150, a communications interface 170. Those skilled in the art will appreciate that the cartridge reader 130 may be practiced in any type of computer operating environment such as personal computers, personal digital assistants (PDA), hand-held devices, portable computers, intelligent pagers, multiprocessor systems, microprocessor-based or programmable consumer electronics, minicomputers, mainframe computers, and the like. Cartridge reader 130 may also be implemented in distributed computing environments where tasks are performed by remote processing devices.

In this exemplary embodiment, imager 140 comprises a solid-state camera 141 with its associated lens 142, a light source 143, a pH sensor 144, a temperature sensor 145, and a cartridge ID sensor 146. Camera 141 connects to a system bus 131 through a camera interface 154, while light source 143, pH sensor 144, temperature sensor 145, and cartridge ID sensor 146 connect to system bus 131 through an analog interface 151. Separable cartridge 110 is typically inserted, connected, attached or otherwise mated to cartridge reader 130.

Cartridge 110, which includes active sites area 111, is detected by cartridge reader 130 when cartridge 110 is inserted into and operatively connected with cartridge reader 130. Upon detecting the presence of cartridge 110, cartridge reader 130 may optionally detect a cartridge ID 113 encoded in or on the cartridge 110. The cartridge ID 113 may be sensed with a cartridge ID sensor 146 or may be optically detected with camera 141. Cartridge ID 113 may be encoded in or on the cartridge 110 by utilizing a bar code, a magnetic strip, or an electronic circuit. Those skilled in the art, however, will appreciate that many other types of cartridge IDs may be encoded in or on the cartridge 110.

Imager 140 responds to changes in active sites area 111. This active sites area 111 typically contains reagents which are exposed to sample 115, whereby changes in the reagents are detected by imager 140. Examples of samples provided to such a active sites area 111 may include, but are not limited to, a biological substance (e.g., blood, urine, etc.) and can be in the form of a liquid, a gas, or a combination of the two. It is further contemplated that samples can also include other liquids and gases when testing non-biological subject, such as the water from a particular river or waste runoff from a manufacturing process.

The ability of an active sites area system 1 to be used for a variety of different types of testing will depend on the nature of cartridge 110. Each cartridge 110 will include an active sites area 111 which includes a plurality of chemically sensitive particles referred to as elements, each of the elements including receptors specific for the desired task. For example, a particular type of cartridge 110 used in medical testing for diabetes may include a number of elements that are sensitive to sugars. Another type of cartridge 110 used in water testing, however, would include different elements, for example, elements specific for pH and metal ions.

By placing the active sites area 111 in the object plane of the focusing lens 142 of camera 141, a change in the elements of active sites area 111 due to the presence of sample 115 is detected optically. This change may include a color change of the elements of active sites area 111. In addition, the temperature and pH of sample 115 are also detected by the temperature sensor 145 and pH sensor 144, respectively. Optionally, one or any combination of cartridge sensor 146, temperature sensor 145 and pH sensor 144 may be integrated into or their respective functions accomplished by the camera 141.

Computer 150 (more generally referred to as processing circuitry which includes the pre-processing functionality discussed earlier) interfaces to imager 140. In general, computer 150 is used to accept test data from the imager 140 and process the test data prior to its transmission out of cartridge reader 130. In the exemplary embodiment, computer 150 comprises a processing unit 152, a memory 153, a camera interface 154 and a sensor interface 151. Memory 153 may include read only memory (ROM) and random access memory (RAM). Note that the functionality of sensor interface 151 may alternately accommodate either analog or digital sensors, resulting in an output that is digital in order to interface with the remainder of the computer 150 components.

Computer 150 may further include a second data storage device that allows the physical introduction or removal of data, information or application programs to or from computer 150. Such a capability may be implemented in the form of a hard disk drive or a magnetic disk drive, to read from or write to a removable disk, or an optical disk drive. The hard disk drive, magnetic disk drive, and/or optical disk drive may be connected to a system bus 131 by a hard disk drive interface, a magnetic disk drive interface, and an optical drive interface, respectively. The drives and their associated computer-readable media provide nonvolatile storage for computer 150. Although the description of computer-readable media above refers to a hard disk, a removable magnetic disk, and a CD-ROM disk, it should be appreciated by those skilled in the art that other types of media that are readable by a computer system, such as magnetic cassettes, flash memory cards, memory sticks, DVD, Bernoulli cartridges, and the like, may also be used in the exemplary operating environment.

Optionally, cartridge reader 130 further includes a global positioning system (GPS) receiver 131 (with its associated antenna 132) and a biometric scanner 133, each of which are attached to computer 150. Using signals from satellites, GPS receiver 131 can pinpoint its current geographic location in the form of location data, such as latitude/longitude coordinates. This functionality can be of particular use in applications such as precision farming, wherein it becomes important to accurately track crop, soil, or water parameters as a function of precise location. Biometric scanner 133 collects data on the biological identification of the subject and/or the user of cartridge reader 130, which may include data relative to voice, eye, handprints, fingerprints and hand-written signatures. Biometric scanner 133 may comprise a voice, retinal, handprint, fingerprint, or hand-writing scanner, however, those skilled in the art will appreciate that many other types of biometric scanners may be utilized.

Input devices are often connected to processing unit 152 through a serial port interface (not shown) that is coupled to system bus 131, but may be connected by other interfaces, such as a parallel port, a game port or a universal serial bus (USB) interface 171. Output or display devices, such as display 134, may also be connected to computer 150 via an interface, such as a video adapter (not shown).

In the exemplary embodiment, computer 150 operates in a networked environment using logical connections to one or more remote computer systems via communications interface 111. Such remote computer systems may be a server, a router, a peer device (i.e. another cartridge reader) or other common network node, and typically includes many or all of the elements described relative to computer 150. The logical connections utilized in the networked environment may include a local area network (LAN) or a wide area network (WAN).

When used in a LAN networking environment, computer 150 may be connected to the LAN through a network interface, such as an IEEE 802.3 Ethernet card 172. However, when used in a WAN networking environment, communications interface 111 typically includes a modem 173 or other means for establishing communications over the WAN such as the Internet. Modem 173, which may be internal or external, is connected to computer 150 via a serial port interface.

A tremendously enabling feature of the present invention is the remote database analysis and interpretation site, referred to as the data service. This is the centralization point at which electronic information transmitted from one or more cartridge readers is collected and stored; optionally associated identifications are confirmed; remote control and access of the individual cartridge readers is enabled; original optical signature information is received from the readers and processed, and billing actions initiated.

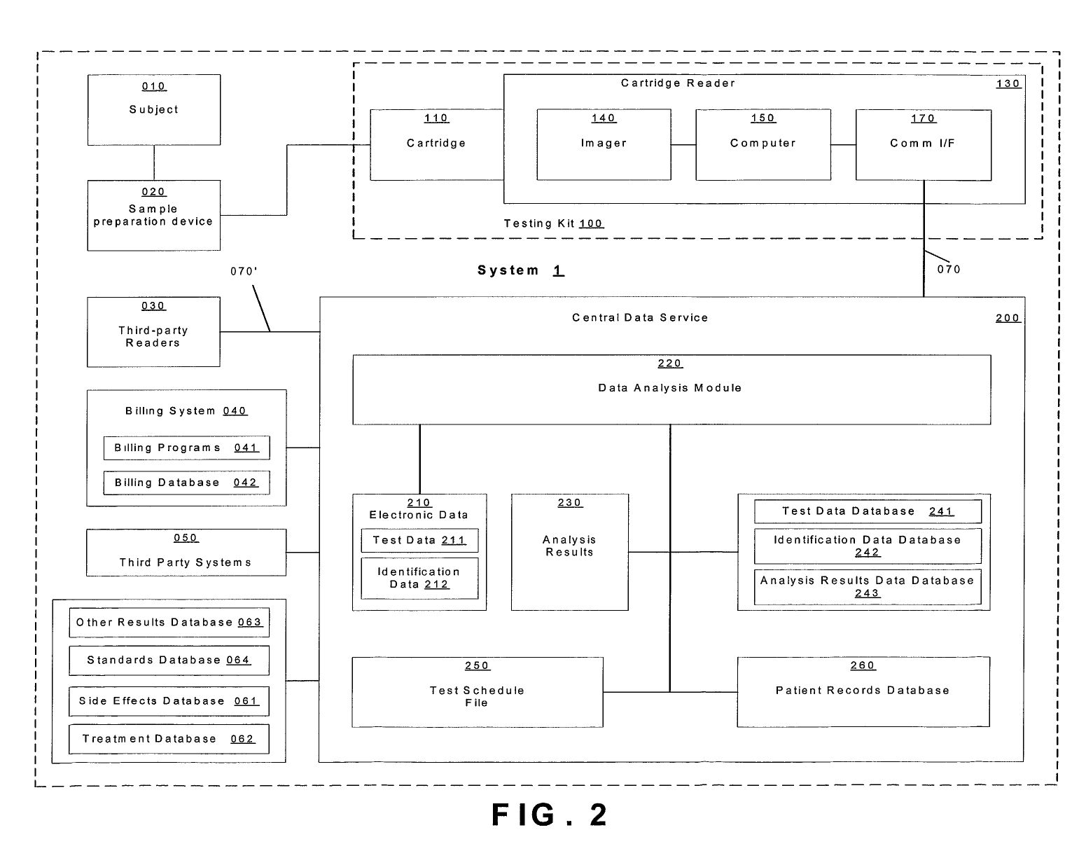

Referring now to FIG. 2, an exemplary system 1 environment for the present invention comprises cartridge reader 130, cartridge 110, and a data service 200. As previously described, cartridge 110 may include a active sites area 111, and the sensor area 111 may be exposed to a sample 115 obtained from subject 010. In some cases, the original sample obtained from subject 010 must undergo some level of processing before it can be introduced to the cartridge 110 as sample 115. This preparation may include the addition of one or more reagents, liquification, temperature treatment, grinding, etc. Some of these features may be directly incorporated into cartridge reader 130 and possibly directly controlled by data service 200 as discussed later. Cartridge reader 130 typically gathers electronic data related to an optically-detectable change in the active sites area 111. Cartridge reader 130 prepares the electronic data for transmission to data service 200, which typically comprises a central server.

The data service 200 exchanges electronic data with cartridge reader 130 in a format supported by data service 200 via a wireless or wireline connection 070. In addition to cartridge reader 130, data service 200 may exchange electronic data with third-party cartridge readers 030 over a wireless or wireline connection 070′. Third-party cartridge readers 030 are devices similar to cartridge reader 130 in that they collect and exchange electronic data with data service 200 in a protocol supported by data service 200. Those skilled in the art will appreciate that various types of communication techniques can be used to provide wireless or wireline transmission, including, for example, infrared line-of-sight, cellular, microwave, satellite, packet radio, spread spectrum radio, LAN, WAN, and Internet communications.

Electronic data exchanged between cartridge reader 130 and data service 200, and third-party readers 030 and data service 200 may comprise electronic data 210, which may be stored in a volatile or non-volatile form within data service 200. Electronic data 210 typically includes (but is not limited to) test data 211 and identification data 212. Test data 211 are related to an optically detectable change in active sites area 111 after a sample 115 has been introduced. Identification data 212 is generally any other data transmitted from cartridge reader 130 that is related to the test being performed and may comprise, for example, at least one of the following: cartridge ID 113, a sample ID, a subject ID, a cartridge reader ID, a cartridge reader operator ID, a time electronic data 210 was gathered, a date electronic data 210 was gathered, a location where electronic data 210 was gathered, a sample temperature, and a sample pH level.

A data analysis module 220 is a coded set of instructions or a program module that analyzes the received electronic data 210 to produce analysis results 230. Test data 211, identification data 212, and analysis results 230 are typically archived in test data database 241, identification data database 242, and analysis results database 243, respectively. A test schedule file 250 may also utilize data service 200 to store test schedules so that reminders and prompts may be issued to a subject in accordance with a test schedule. Patient records are normally stored in a patient records database 260 to the extent that patient records are necessary in the analysis of the electronic data 210.

If a diagnosis is to be issued when analysis results 230 are presented to third-party systems 350, data utilized in formulating the diagnosis are stored in other databases 060. These databases include but are not limited to side effects database 061, treatment database 062, results databases representing comparable patients/subject 063, and standards databases 064 which contain information for acceptable ranges of the subject characteristics being tested. Standards databases 064 represent an important tool if the present invention embodiment involves the actual diagnoses of a patient based upon the electronic information generated. It is further anticipated that data service 200 may be connected through network 070 so that it is capable of accessing other databases (not shown) with medical treatment, drug interaction information, publicly and privately available information regarding the sample (e.g., historical data on the sample).

Third-party systems 050 to which results, billing, and the like may be directed, may include the systems of the test subject, a cartridge reader operator, a health care or medical provider (such as a doctor, a nurse, clinic, etc.), an insurance company, a government agency, or financial compensator. Those skilled in the art, however, will appreciate that many other third-party systems 050 may be utilized.

Billing transactions are initiated utilizing billing system 040, which comprises billing programs 041 and billing databases 042. A billing transaction should be interpreted to mean any kind of billing decision or billing event, including reimbursement, such as flagging a record associated with the billed party. While illustrated as a separate computer system connected to data service 200, it is contemplated that data service 200 may include such billing functions.

Billing transactions may be initiated at a variety of stages or as the result of specific events, such as when electronic data 210 are communicated to data service 200; when analysis results 230 are presented to third party system 050; when analysis is performed by data analysis module 220; or when data are archived in test data database 241, identification data database 242, or analysis results database 243. Other billing transactions may occur when the analysis results are provided to other entities or when test kits (including testing cartridges such as cartridge 110) are sold to vendors, subjects or other third parties.

A billing transaction may involve one or more of the following: a test subject, a cartridge reader operator, a health care provider, a physician, an insurance company, a government agency, or a financial compensator. Those skilled in the art, however, will appreciate that many other persons or entities may be involved in billing transactions.

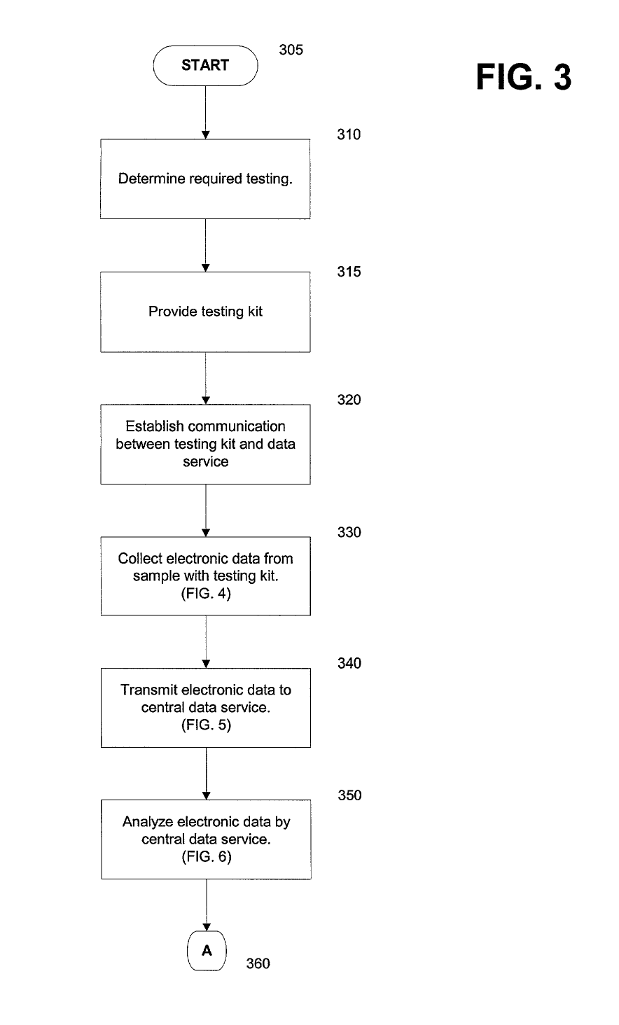

FIG. 3 is a flow chart setting forth the general stages involved in an exemplary method 300 for gathering data on a sample utilizing a active sites area system and for analyzing the sample using a central data service or centralized server. Such a method may be used, for example, for: point-of-care (POC), point-of-testing (POT) medical diagnosis; on-site monitoring of process control applications; military intelligence gathering; environmental monitoring; food safety testing; and the like.

For example, cartridge reader 130 may be implemented as a centralized unit such that more than one subject or operator may utilize it. In this case, cartridge reader 130 may be a desk-top or table-top model intended to be used at a designated site. With such a stationary cartridge reader 130, cartridge 110 is brought to the cartridge reader 130. The size of stationary cartridge reader 130 may be larger than a hand-held version. In this embodiment, cartridge reader 130 may be located in a pharmacy so that at-home patients can bring cartridge 110 to cartridge reader 130 to be read and uploaded to data service 200 for interpretation. Correspondingly, cartridge 110 may be dispensed by prescription and used at home. Cartridge 110 could then be returned to the pharmacy and subsequently read by cartridge reader 130 and uploaded to data service 200.

The implementation of the stages of method 300 in accordance with an exemplary embodiment of the present invention will be described in greater detail in subsequent figures. Exemplary method 300 begins at starting block 305 and proceeds to subroutine 310 where it is determined what subject 010 characteristics are to be tested and what testing kit is therefore required. The exemplary method 300 then accomplishes subroutine 315 where the required testing kit is introduced to the subject 010. At the time of testing, the cartridge reader 130 and the data service 200 exchange identification and administrative information through subroutine 320. Within subroutine 320, data service 200 may provide operational instructions to the cartridge reader 130 such that the actual control of the testing is accomplished at the data service 200 location. Such control may be implemented through the providing of instructions to the testing kit 130 operator in the form of prompts or, alternatively, the data service 200 may assert direct control over the operation of the testing kit 130, thereby automatically performing the required testing without potential influence or interference from the testing kit 130 location.

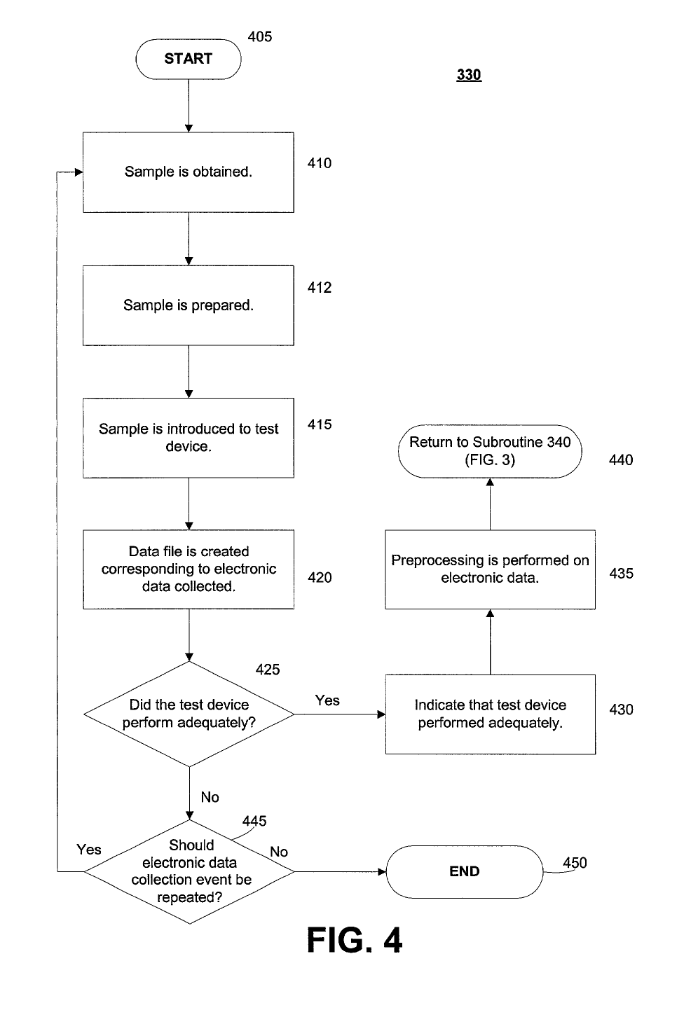

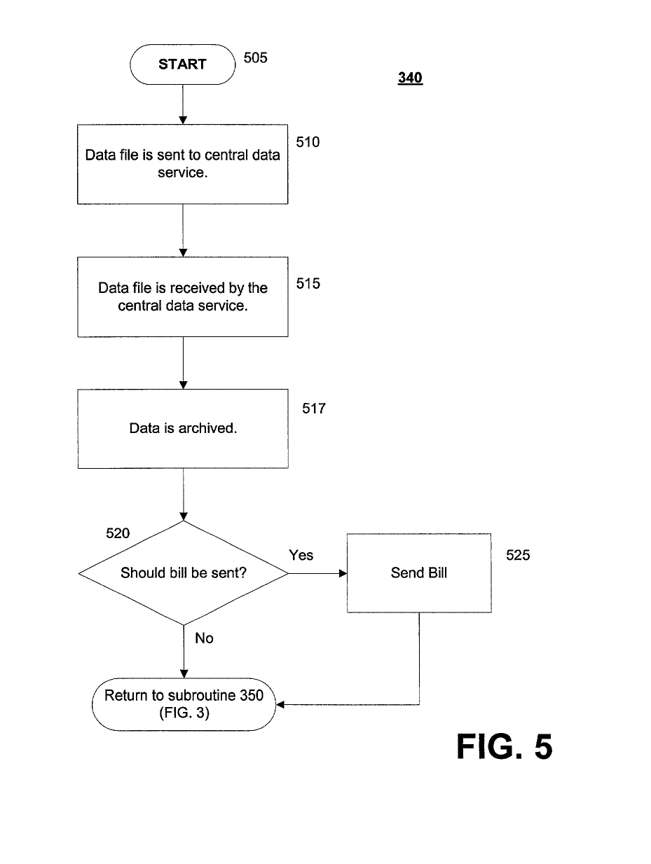

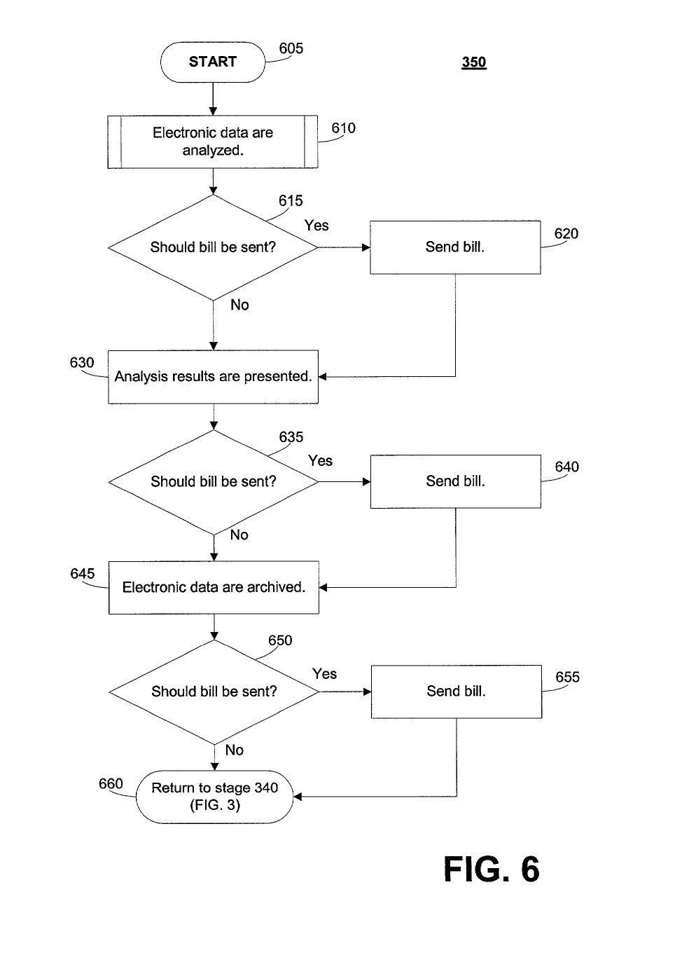

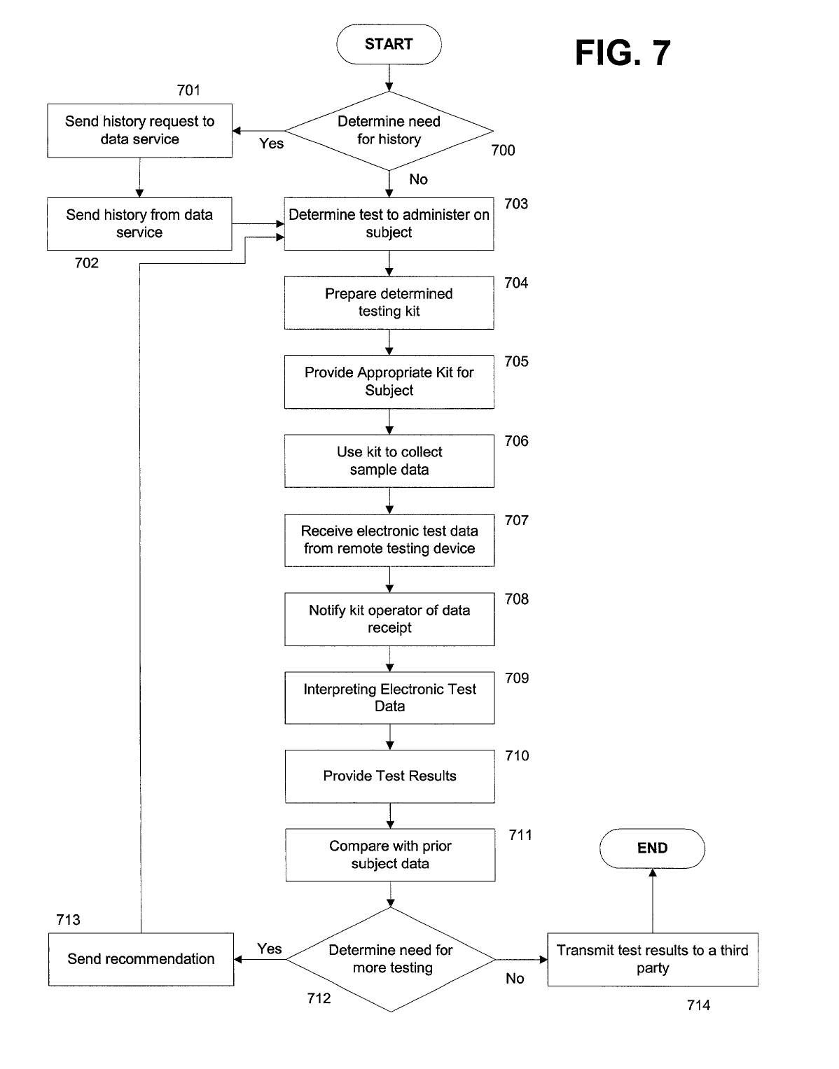

The next subroutine 330 is where electronic data 210 are collected on sample 115 with cartridge reader 130 using active sites area 111. The stages of subroutine 330 are shown in FIG. 4 and will be described in greater detail below. From subroutine 330, where electronic data 210 are collected on sample 115 with cartridge reader 130 using active sites area 111, exemplary method 300 continues to subroutine 340 where electronic data 210 are sent to data service 200. The stages of subroutine 340 are shown in FIG. 5 and will be described in greater detail below. Once electronic data 210 are sent to data service 200 in subroutine 340, exemplary method 300 continues to subroutine 350 where electronic data 210 are analyzed by data service 200. The stages of subroutine 350 are shown in FIG. 6 and will be described in greater detail below. After electronic data 210 are analyzed in subroutine 350, exemplary method 300 moves to stage A where the analyzed electronic data are used and further processed to pre-screen a subject, as discussed in more detail below with regard to FIG. 7.

FIG. 4 illustrates the exemplary subroutine 330 from FIG. 3, in which electronic data 210 are collected on sample 115 with cartridge reader 130 using active sites area 111. Referring now to FIG. 4, exemplary subroutine 330 begins at starting block 405 and advances to stage 410 where an original sample from subject 010 is obtained. This original sample may comprise a bodily substance such as blood, urine, fecal matter, or saliva. This sample is not limited to a biological substance, and it may be in the form of a liquid, a gas, or a combination of the two. Those skilled in the art will appreciate that this original sample many comprise many other substances in various forms.

Next, this original sample undergoes a preparation phase 412 where it is subjected to chemical, temperature or other physical conditioning to prepare it for introduction to the cartridge 110. At this point, the original sample has been transformed into the sample 115 which is then introduced to cartridge reader 130 and cartridge 110 in exemplary subroutine 415. In the exemplary embodiment, cartridge ID 113 is detected when cartridge 110 is initially inserted, attached, connected or otherwise mated to cartridge reader 130. It is contemplated that cartridge ID 113 may be detected by a variety of conventional electronic identification methods, such as, for example, optically reading a bar code on cartridge 110, reading an electronic logic circuit placed within cartridge 110, or reading a magnetic tape placed on cartridge 110.

Cartridge ID 113 may be used for a number of purposes, such as to indicate the source of the cartridge 110, a lot number when cartridge 110 was manufactured such that a corresponding expiration date can be determined, the appropriate data analysis module 220 to employ, or where to route the resulting data. Similarly, cartridge ID 113 may direct data service 200 as to where results, bills, earned fees, and the like should be directed. For example, a hospital could purchase a plurality of cartridges that are identified to the hospital. When these cartridges are used and their cartridge IDs are read, data service 200 will have sufficient information as to where results, bills, and the like should be directed.

After cartridge ID 113 is detected, biometric data for the operator may be acquired. Biometric scanner 133 collects data on the biological identification of a person, which may include data relative to the voice, eye, handprints, fingerprints and hand-written signatures. While biometric scanner 133 is a fingerprint scanner in the exemplary embodiment, biometric scanner may comprise a voice, retinal, handprint, fingerprint, hand-writing scanner, or any other types of biometric scanner.

In the exemplary embodiment, the biometric data for the operator are typically sent to data service 200 via connection 070. Data service 200 then responds with approval data. For example, data service 200 may include a database of approved operators. Once biometric data have been collected on the operator, these data may be electronically checked against a database of approved operators. Depending on a match for the biometric data on the operator, data service 200 may communicate the approval or non-approval of the operator back to cartridge reader 130. Typically, if the operator is not initially approved, biometric data are re-acquired.

Biometric data from the biometric scanner 133 may also be collected from the subject which, in the case of a medical application, more accurately identifies the patient and corresponding improves overall system reliability.

Once approval is received, other identification information related to the electronic data collection event is gathered, such as subject ID, sample ID, location ID, cartridge reader ID, time, and date. Subject ID and sample ID may be obtained using biometric scanner 133. Location ID may be obtained manually by operator input or by pinpointing the current location of cartridge reader 130 by using GPS receiver 131 to triangulate satellite signals to determine location. Cartridge reader ID, time, and date may be obtained from computer 150, which contains an internal clock and calendar. Cartridge reader ID may be stored in memory 153 of computer 150 and may be retrieved from memory 153 when needed.

In the exemplary embodiment, imager 140 then typically acquires the initial image utilizing camera 141 and sends the initial image to data service 200. Data service 200 determines testing information based upon the initial image and sends that information back to the cartridge reader 130. The initial image received by the data service 200 is used to identify a cartridge type for cartridge 110. Once identified, the data service 200 looks up the necessary test information associated with the cartridge type for cartridge 110. This information is transmitted to cartridge reader 130 via a wireless or wireline connection 070. Cartridge reader 130 can use this information to select and adjust light source 143, and to initialize the test results timers (i.e., time to wait for chemical reaction to complete) and wash timers (i.e., time to wait between applying the analyte and applying the wash, if necessary).

Once cartridge reader 130 has received the testing information and is properly configured, active sites area 1 11 is exposed to sample 115. Active sites area 111 contains reactants which are exposed to sample 115. The ability of an active sites area system to be used for a variety of different types of testing will depend on the nature of the cartridge 110 used. Each cartridge 110 will include an active sites area system which includes a plurality of chemically-sensitive particles referred to as elements, each of the elements including receptors specific for the desired task.

After a temperature and a pH value for sample 115 are acquired, it is determined if the next step in sample 115 processing can be accomplished or if additional sample preparation is needed, such as applying a wash or instructing the cartridge reader 130 to heat or cool the sample 115, respectively. Wash may be desired to introduce a cleaning solution (a wash) allowing chemical reactions to take place and/or be observed on elements of active sites area 111. If wash is desired, a wash is applied to active sites area 111. If not, cartridge reader 130 acquires a final image of a detected change in a multiple analyte senor area 111. This change is detected optically utilizing camera 141 to construct the final image of which the test data 211 are comprised. At the same time, calibration data are detected optically utilizing camera 141 and are included in the final image.

A data file is then created within data service 200 corresponding to electronic data 210 at stage 420. As stated previously, electronic data 210 typically includes but is not limited to test data 211 and identification data 212. Test data 211 represents a detected change in an active sites region as normally detected utilizing camera 141. An image or a series of images is collected utilizing camera 141 representative of the optical change within the cartridge. This image or series of images could be comprised of before and after images (i.e. before and after the introduction of the test sample) or could be comprised of a timed sequence of images depicting a time rate of change reaction within the cartridge. Depending upon the specific algorithm utilized to evaluate the resulting optical change in the cartridge, some level of preprocessing may be performed prior to transmitting the data to the central processing center. Such preprocessing could include the compression of the images collected by deleting portions of the image that do not contain useful optical information or by performing some initial calculations. There is a trade-off between the desire to limit the volume of data to be transmitted for bandwidth considerations and the desire to store as much of the originally captured data as possible in the central data center.

At the same time, calibration data may be optically detected with camera 141 and may be included in the final image. This calibration data may be generated from portions of the cartridge within the field of regard of the camera dedicated to the creation of controlled image elements not dependent upon the chemical interactivity of the cartridge-based analytes with the sample provided. Such image elements introduced as part of the original images allows the final analytical algorithm to account for potential variations within the camera and associated hardware when performing the final evaluation of the optical changes apparent within the cartridge.

Identification data 212 are the information associated with the test data and normally include at least one of the following: cartridge ID 113, sample ID, subject ID, cartridge reader ID, cartridge reader operator ID, time test data 211 was gathered, date test data 211 was gathered, location where test data 211 was gathered, sample temperature, and sample pH level. Sample ID and subject ID may be obtained using biometric scanner 133 and the location where test data 211 was gathered may be obtained using GPS receiver 131.

Once the data file is created, a determination is made at step 425 whether cartridge reader 130 performed adequately. Such determination could be made based on the evaluation of the calibration data contained within the data file as to whether or not the values contained within that calibration data were within predetermined limits of acceptable operation. Also, at the data service, the data file received can be compared to all other data files received for that specific cartridge of series of cartridges such that any significant deviation from the typical result can be identified and the operator immediately notified of a potentially suspicious result. If the cartridge reader 130 performed adequately, exemplary subroutine 430 advances to stage 430 where it is typically indicated that cartridge reader 130 performed adequately. However, if cartridge reader 130 did not perform adequately, exemplary subroutine 425 advances to decision block 445 where it is determined if electronic data collection event should be repeated. If so, exemplary subroutine 445 advances to stage 410 where sample 115 is again obtained and stages of exemplary subroutine are repeated as described above. If the electronic data collection event should not be repeated, the process is halted due to a malfunction in the data collection process, caused either by a failure in cartridge reader 130 or a failure in the application of the sample 115 to cartridge 110, or incorrectly mating cartridge 110 to cartridge reader 130.

When performing a calibration check, certain “null” or “control” elements of the active sites area 111 may be considered. These elements have a fixed color which may be determine by cartridge reader 130 from the cartridge ID 113. For example, cartridges with certain cartridge ID numbers may have specifically colored null elements. When the color of the null elements is determined from the cartridge ID, this information can be compared against the color of the null elements taken from the final image. By comparing the two data, the calibration of cartridge reader 130 may be determined.

When cartridge reader 130 has performed adequately, exemplary subroutine 430 advances to stage 435, where preprocessing is performed on electronic data 210. This preprocessing may include parsing electronic data 210, formatting electronic data 210, or encrypting electronic data 210 and including this in electronic data 210.

One example of parsing electronic data 210 may include deleting portions of the test data 211 that do not include image information of the responsive elements in the active sites area 111. When the final image is taken of active sites area 111, this image contains a space between the individual elements of active sites area 111. Because the space between the individual elements of active sites area 111 contains no useful information, a portion of the image corresponding to the space may be deleted from the test data 211.

It is contemplated that conventional cryptographic standards may be used to encrypt the data. Public key cryptography software from Pretty Good Privacy, Inc., (PGP) of San Mateo, Calif., (www.pgp.com) may be utilized in this embodiment. PGP was developed by Phil Zimmermann, founder of the company, and it is based on conventional RSA cryptographic method. A version for personal, non-business use is available on various Internet hosts.

After preprocessing is performed on electronic data 210 in exemplary subroutine 435, exemplary subroutine 330 continues to stage 440 and returns to stage 340 of FIG. 3. Turning now to FIG. 5, the exemplary subroutine 340 from FIG. 3 is more specifically described, in which electronic data 210 are sent to central data service 200. Exemplary subroutine 340 begins at starting block 505 and advances to stage 510 where the data file is sent to central data service 200. In the exemplary embodiment, this is accomplished over a wireless or wireline connection 070, where the data file is then received by central data service 200 in stage 515. At this point, the electronic data 210 may be stored as shown in step 517 for later retrieval and processing.

Once data file is received, exemplary subroutine 340 advances to decision block 520, where it is determined if a bill or invoice should be sent. If it is determined that bill should be sent at decision block 520, exemplary subroutine 340 advances to stage 525 where bill is sent. The bill may be sent to the test subject, a test device operator, a health care provider, a physician, an insurance company, a government agency, or a financial compensator. If, at decision block 520, it is determined that a bill should not be sent, exemplary subroutine 520 advances to stage 530 and returns to stage 350 of FIG. 3. Similarly, once a bill is sent, as shown at stage 525, exemplary subroutine 525 advances to stage 530 and returns to stage 350 of FIG. 3.

Turning now to FIG. 6, the exemplary subroutine 350 from FIG. 3 is more specifically described in which electronic data 210 are analyzed by central data service 200. Exemplary subroutine 350 begins at starting block 605 and advances to exemplary subroutine 610 where electronic data 210 are analyzed. In the exemplary embodiment, an appropriate analysis is determined based upon identification data 212 associated with test data 211. For example, if identification data 212 indicate that cartridge 110 was for medical testing of diabetes, then the appropriate analysis may include a diabetes analysis.

Furthermore, a diagnosis based upon analysis results 230 may be provided by on-line physicians or technicians performing real time analysis of the analysis results or by using an expert system. Determining a proper diagnosis may comprise correlating analysis results 230 with treatment data contained in treatment database 062 or with side effects data contained in side effects database 061. An expert system is an artificial intelligence (A.I. or AI) application that uses a knowledge base of human expertise for problem solving. Its success is based on the quality of the data and rules obtained from the human expert. In practice, expert systems perform both below and above that of a human. It derives its answers by running the knowledge base through an inference engine, which is software that interacts with the user and processes the results from the rules and data in the knowledge base. The knowledge base in this embodiment comprises treatment database 062 and side effects database 061. Examples of uses for expert systems other than for medical diagnosis are equipment repair, investment analysis, financial, estate and insurance planning, vehicle routing, contract bidding, production control and training.

Next, the calibration of cartridge reader 130 is typically checked. Similar to the preprocessing stage of FIG. 4, the calibration can be checked at central data service 200. When performing a calibration check, certain “null” or “control” elements of active sites area 111 are taken into consideration. In the exemplary embodiment, these elements have an expected color signature which is determined by cartridge reader 130 from cartridge ID 113. For example, cartridges with certain cartridge ID numbers may have specifically colored null elements. When the color of the null elements is determined from the cartridge ID, this information can be compared against the color of the null elements taken from the image. By comparing the two data, the calibration of cartridge reader 130 may be determined.

Next, a determination may be made as to whether a schedule of tests is completed. If the schedule of tests is not complete, a reminder may be issued. This reminder may be issued to the test subject, the test device operator, a health care provider, a physician, an insurance company, a government agency, or a financial compensator. However, if the schedule of tests is complete, no reminder is necessary.

Referring again to FIG. 6, once electronic data 210 are analyzed in exemplary subroutine 610, exemplary subroutine 350 advances to decision block 615 where it is determined if a bill should be sent to similar recipients as noted above. If a bill should be sent at decision block 615, exemplary subroutine 350 first advances to stage 620, where a bill is sent, and second advances to stage 630. If it is determined, however, that a bill should not be sent, exemplary subroutine 350 immediately advances to stage 630 where analysis results 230 may be presented providing some predetermined criteria is met, such as confirmed operator or subject ID, billing/account history, or specific health-care provider instructions. As discussed below, the analysis results 230 may be presented to the test subject, a test device operator, a health care provider, a physician, an insurance company, a government agency or financial compensator.

Once analysis results 230 are presented, a determination is made at stage 635 if a bill should be sent. If a bill should be sent, exemplary subroutine 350 advances to stage 640, where the bill is sent to one or more of the typical recipients mentioned above and the electronic data 210 are archived at stage 645. However, a bill should not be sent, exemplary subroutine 350 advances directly to stage 645, where electronic data 210 are archived. Specifically, test data 211, identification data 212, and analysis results 230 or a subset thereof may be selectively archived in test data database 241, identification data database 242, and analysis results database 243, respectively. Next, a determination is again made if a bill should be sent at block 650. If so, a bill is sent at stage 655. If not, exemplary subroutine 350 advances directly to stage 660 and returns to stage 360 of FIG. 3.

To further elaborate upon the potential processing to be accomplished at the data service 200, an exemplary method for remote testing with centralized correlation is described with an embodiment of the present invention. As described previously, a testing kit 100 is provided for a subject and is comprised of cartridge reader 130 having a replaceable cartridge 110, which may be tailored for the desired test. Once a subject has collected a sample 115 and placed it in active site area 111, electronic data are collected for sample 115, and these electronic data are then sent to data service 200 for processing.

The electronic data are analyzed according to the desired test, and test results are provided. Test results may optionally be provided to the subject or a third party, such as a medical provider, regulatory agency, or insurer.

Test results may advantageously be correlated with a secondary data set. The term “correlating” should be broadly interpreted to mean finding any relationship between different parts or elements of the test results or between the test results and the secondary data set (e.g., historical test data, treatment data, side effect data, etc.), whether correlation consists of searching for suspected relationships or using algorithms, tools, or techniques that identify previously unsuspected data relationships. In the exemplary embodiment, data service 200 may query one or more other databases 060, such as a side effects database 061 or a treatment database 062, as part of the correlation activity. This activity seeks to find similar trends or distinct similarities between the electronic data from the cartridge reader 130 and archived data. In one aspect, the correlation activity may access a treatment database when correlating the test data. In general terms, a treatment database is archived data on a variety of treatments available to help the subject. Typically, such a database provides potential treatments when queried or compared with a given set of test data.

The result of such correlation may reveal possible treatments for the subject. For example, this is helpful when the subject is a medical patient and an initial review of the test results by a medical provider (e.g., a doctor, nurse, laboratory technician) is less than conclusive as to the medical condition of the subject and as to potential treatments. The results of such correlation may identify one or more similarities with archived data, such as the treatment data.

When the accessed database is a side effect database, the information being correlated with the test results may help identify one or more similarities with known drug interactions or side effects due to drugs being prescribed and taken by the subject.

When the accessed database is simply historical test data on the subject (e.g., historical blood sugar levels, a history of bacterial information gathered from the trunk mucus of an elephant, historical water levels for a particular lake, historical plankton count in an area of the ocean known for whale breeding, etc.), trends from such compiled information can be identified and compared with the present test results. Again, this may provide further insight into what is going on within the subject (e.g., a human body, an animal, a lake, an ocean, etc.) due to correlating the present test data with the other data.

In other embodiments, the correlation activity may be performed among elements of the same data set with or without queries to other databases 060. In this way, conclusions may be drawn by comparing the two sets of data.

These conclusions or simply the results of the correlation may be provided to the test subject or third parties, such as a medical provider, regulatory agency, or other company. Providing such results or conclusions about the results is typically accomplished via an electronic message sent from central data service to the appropriate recipient. However, the recipients may be notified of these results or conclusions via flagged records, facsimile transmissions, or other known notification means. In the medical environment, providing these results is often in the form of notifying the test subject or third party about one or more possible treatments, discovered side effects or other relationship information.

The present invention enables a method for pre-screening a subject using a remote testing device capable of detecting/measuring multiple substances in an administered sample. More particularly, the present invention provides for testing and data management services when screening a subject prior to an office visit utilizing a customized test kit, which includes a testing kit described earlier.

Consistent with embodiments of the present invention, methods and systems for pre-screening a subject are disclosed. The methods and systems allow a subject, such as a medical patient, to perform one or more screening tests without the intervention of a testing professional, such as a medical care-giver or other medical provider. In this way, the subject may use a set of preliminary results to focus the subject's need for medical intervention. For example, a pre-screening test may produce results whereby a general medical professional is bypassed in favor of the services of a medical specialist. Furthermore, results of the pre-screening test may be provided to a medical provider in preparation for an office visit so that the office visit is enhanced and becomes a much more efficient use of both the subject's and the provider's time.

Other embodiments of the present invention include examples from both the veterinary sciences field as well as the agricultural and geological fields. In the case of the former, the present invention may be employed to accomplish pre-screening for large animals where it is impractical to transport the animal to a veterinary clinic to determine it's need for care or costly to have a veterinarian screen the animal on-site. Using the present invention for this application, an on-site user can use a supplied test kit to take the sample required from the animal, apply it to the cartridge(s) provided, insert the cartridge into the diagnostic device provided or already available to the user, and transmit the collected data via modem or internet (as examples) to a veterinarian's office, clinic or centralized diagnostic center. After arriving at its destination, the data is analyzed, the animal is diagnosed, and a recommended course of action is provided to the on-site user. This recommendation might include the application of a wide variety of medicinal treatments or it might suggest further evaluation, either through the application of additional diagnostic test kits or by the on-site intervention of a veterinarian or technician.

An embodiment that has agricultural and environmental implications has the pre-screening test kit supplied to an on-site user who is determining local conditions. This determination can be made using either on a soil or a water sample and is based on the amount or presence of one or more elements sensed by the multiple analyte diagnostic device contained in the kit. Here again, the sample is applied to the cartridge provided which is inserted into the diagnostic device and the collected data is then transmitted back to a centralized laboratory where the data is evaluated. The determination is then made as to whether further testing is warranted or if the diagnosed conditions indicate a pre-determined course of action, such, as in the agricultural application, the administering of fertilizers or other chemicals to the area tested, or, for the environmental application, the need for monitoring specific pollutants or other chemicals.

In the context of the above description regarding sampling, gathering electronic data and centrally analyzing such data, an exemplary method of pre-screening a subject using a remote testing device is described as shown in FIG. 7 consistent with an embodiment of the present invention. With reference to FIG. 7, a determination is made as to whether or not the patient records database 260 or one of the other available databases 060 should be examined. If the determination is made that such an examination is required, a request is sent to the data service via step 701 and the requested data is sent in step 702. Stage 703 is then entered. I the result of stage 700 is negative, stage 703 is entered directly. Often, the determination of stage 703 would be made by a third party, such as a doctor, a doctor's office, a hospital, a nurse, or a nursing home, for example. Stage 703 may also comprise a review of a history of the subject to determine which test should be administered. For example, patient records database 260 of data service 200 may be queried to provide the medical history of the subject. The determination of what test to administer on the subject could then be based on the reviewed history. For example, if patient records database 260 revealed that a subject was borderline diabetic or has a family history of heart disease and high blood pressure, stage 703 may result in the determination that a blood sugar test should be administered to the subject prior to having an office visit.

A remote testing kit 100 is then defined in stage 704 and provided for the subject at stage 705. Typically, the kit 100 comprises a selected cartridge 110 and may, in some circumstances, also include a remote testing device 105. In an exemplary embodiment, the cartridge 110 is selected based on the determination made at stage 703. The kit 100 may be customized prior to sending it depends on the text and the history of the subject. Moreover, providing kit 100 for the subject may comprise mailing or otherwise delivering the kit to the subject. Once the subject has received and administered the test as shown in stage 706, electronic test data are received from the remote testing device, as shown in stage 707. These data may be received at data service 200 for further processing, for example. The data service 200 may, at this point, send a notification to the subject or testing kit operator as shown in stage 708 that the data was received.

Once received, the electronic test data may be interpreted, as shown at stage 709. Moreover, in stage 710, results acquired from the test data may be provided. Test results may then be transmitted to a third party, as shown at stage 710. These results may be compared to previous testing results as shown in stage 711 and a determination is made in stage 712 as to the potential need for additional testing. If no further testing is needed as determined in stage 712, the results are transmitted to a third party in stage 714. If additional testing is indicated, a recommendation for that testing is transmitted via stage 713. The third party discussed here may comprise, for example, a doctor, a doctor's office, a nurse, a hospital, or a nursing home. In an exemplary embodiment, the data interpretation stage 709, providing results stage 710, and transmitting results stage 714 may be performed by data service 200.

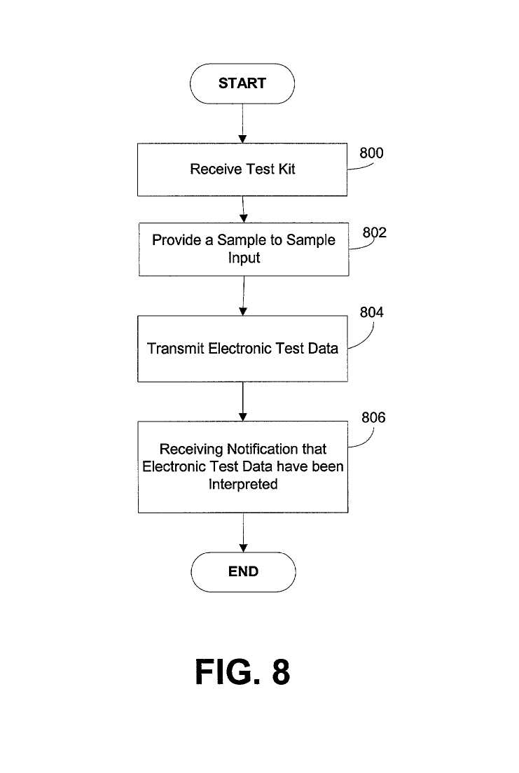

Turning now to FIG. 8, the exemplary method of the present invention is described from the perspective of a test subject. At stage 800, the test subject receives a test kit 100. Typically, the test kit 100 may be mailed or otherwise delivered to the test subject as a means of facilitating pre-screening. The test subject then, at stage 802, provides a sample 115 to the sample input on the cartridge, namely, to the active sites area 111. Sample 115 may comprise, for example, a bodily substance such as blood, urine, fecal matter, or saliva. Once cartridge reader 130 has collected the information from sample 115 and performed the required preprocessing of that information, electronic test data are transmitted from the cartridge reader 130, as shown at stage 804. In an exemplary embodiment, the test data are transmitted to data service 200 for interpretation and processing. Once data are analyzed at data service 200, the test subject may receive notification that the electronic test data have been interpreted, as shown at stage 806.

The present invention further includes provisions for prepaid testing using a remote testing device capable of detecting/measuring multiple substances in an administered sample. More particularly, embodiments of the present invention provides for testing and data management services for prepaid testing utilizing a customized test kit, which includes a multiple analyte diagnostic cartridge reader.

Consistent with embodiments of the present invention, methods and systems for prepaid testing are disclosed. The methods and systems allow a subject, such as a medical patient, to acquire a customized prepaid test kit comprising the remote testing device and a replaceable cartridge or set of cartridges and to generate medical data which can be transmitted to a medical care-giver or other health care provider. In accordance with embodiments of the present invention involving a medical patient, a subject generally provides a sample, which is input to a test kit. In the example of medical uses of the current invention, the sample may comprise a bodily substance, such as blood, urine, fecal matter, or saliva, for example. A prepaid analysis fee is incorporated into a price for the customized kit so that, by buying a kit, a subject has prepaid all fees associated with having the data taken from the sample in question, having the data received and analyzed by the central server and for having the associated diagnosis or results transmitted to the subject, transmitted to a third party or archived. In this way, billing systems and methods respecting diagnostic testing may be streamlined and overall use of remote testing devices may become easier and more attractive to users of such remote testing devices. The focus of this portion of the application is on the aspect of how to provide and utilize such prepaid test kits.

Other embodiments of the present invention include examples from both the veterinary sciences field as well as the agricultural and geological fields. In the case of the former, the present invention may be employed to accomplish prepaid testing of animals where a customized test kit as described previously for the medical patient application is supplied. Using the present invention for this application, an operator can follow supplied instructions to take the sample required from the animal, apply it to the cartridge(s) provided, insert the cartridge into the diagnostic device provided or already available to the user, and transmit the collected data via modem or internet (as examples) to a veterinarian's office, clinic or centralized diagnostic center. After arriving at its destination, the data is analyzed, the animal is diagnosed, and a recommended course of action is provided to the cartridge reader user. This recommendation might include the application of a wide variety of medicinal treatments or it might suggest further evaluation, either through the application of additional diagnostic test kits or by the on-site intervention of a veterinarian or technician. The prepaid fee for the customized test kit includes the fees associated with the receipt, analysis, reporting or storing of the generated data.

An embodiment that has agricultural and environmental implications has the customized test kit supplied to a user who is determining local geological conditions. This test may be performed using either on a soil or a water sample and is based on the amount or presence of one or more elements sensed by the multiple analyte diagnostic device contained in the kit. Here again, the sample is applied to the cartridge provided which is inserted into the diagnostic device and the collected data is then transmitted back to a centralized laboratory where the data is evaluated. The determination is then made as to whether further testing is warranted or if the diagnosed conditions indicate a pre-determined course of action, such, as in the agricultural application, the administering of fertilizers or other chemicals to the area tested, or, for the environmental application, the need for monitoring specific pollutants or other chemicals. As in the previously described embodiments, the cost of this customized kit includes prepaid fees associated with the receipt, analysis, reporting or storing of the generated data.

Referring again to FIG. 7, an exemplary method of prepaid testing using a remote test device is illustrated. In stage 703, a test kit is defined for a subject. The testing kit 100 typically comprises a replaceable cartridge 110 related to a desired test and may also include the cartridge reader 130 as well. The testing kit 100 may be customized for a particular type of purchaser, such as for nurses in a hospital that will be testing blood for various chemicals or for biologists that will be testing a body of water for waste chemicals.

A prepaid analysis fee is incorporated into a price for the testing kit 100. For purposes of this disclosure, the term “price” should be broadly interpreted to mean any kind of price, such as a wholesale price, a manufacturer's suggested retail price (MSRP), or an actual retail price, for example. Moreover, a price may be determined based on, by way of example but not limitation, materials costs, manufacturing costs, labor costs, marketing costs, analysis fees, profit margins, and the like. Some cartridges may have incorporated tests that, by their very nature and the chemicals and processing required for their manufacture, are more expensive than others. Similarly, some assays may require additional complexity or controls built into the cartridge thereby making those units more expensive that the “typical” implementation. Hidden costs behind the sell of these cartridges, such as liability insurance, could also vary depending upon the tests performed and the potential liability impacts of an associated mis-diagnosis. The amount and complexity of processing by the central server and the number of recipients of the resulting diagnosis or data will also impact the determined pricing.

In the exemplary embodiment, the prepaid analysis fee typically represents a charge for accessing the central data service, uploading the kind of electronic data associated with the replaceable cartridge in the kit, and analyzing the transmitted electronic test data. In stage 705, it is further contemplated that another fee for sending the kit to the subject may also be incorporated into the price for the kit.

One of the important aspects regarding the prepaid analysis fee and the other fees associated with the test kit is that they are additional but bundled as incorporated costs. This feature helps to resolve many of the confusion issues apparent when users attempt to navigate the complexities of conventional laboratory billing fees, and can help avoid the legislative problems associated with fee splitting. Incurring a single cost at purchase advantageously becomes a one-time event for all test-related expenses, including for all types of recipients and all levels of reporting of the test results. Those skilled in the art will appreciate that the precise amount of such fees will vary depending on the complexity of the tests to be run on the central data service as well as other economic competitive factors.

The kit may be sold, tendered, or otherwise vended to a subject as part of stage 705. In the exemplary embodiment, the subject may be targeted by the testing kit 100 manufacturer to be one of a predetermined type of purchaser. For example, the subject purchasing the kit may be the test subject themselves (e.g., a patient of a doctor) or a particular kind of operator of the remote test device (e.g., a scientist that is part of a governmental regulatory agency that tests water and chemicals for compliance with government regulations). Optionally, the kit may be sold or otherwise vended to a third party to ultimately be distributed to the subject. Similarly, any kind of third party entity may pay the fees for the kit. In the exemplary embodiment, the third party may comprise a medical provider (such as a doctor, a nurse, or a hospital), an insurer, a regulatory agency, or a company that is sponsoring the testing.

Once the subject administers the desired test, electronic test data are received from the remote testing kit 100 at stage 707. As described above, data may then be analyzed at the data service 200, as shown in stage 709. In the exemplary embodiment, electronic test data 210 are received within memory on the data service 200. The appropriate testing is determined from the data and executed on the data yielding test results, which are typically stored on the central data service.

Finally, in stage 710, the test results are prepared for delivery to an appropriate recipient. The appropriate recipient may comprise, for example, the subject, a third party, or a medical provider. In one embodiment, the subject or test device user may select the appropriate recipient via instructions sent from the test device to the central data service. In another embodiment, the kit itself is purchased with a designated recipient for the test results. Information sent as part of the electronic test data to the central data service may indicate that the test results are to be sent to the designated recipient. In such a situation, the test results are then transmitted to the designated recipient.

If more than one test is to be performed after receiving the electronic test data, the price of the kit is normally higher than if only one test is done. Further, the format of the test results can vary according to the price of the kit. For example, if the test results from an exemplary test kit involved a simple response (e.g., Pass or Fail on a screening drug test performed on a subject's urine), the fee apportioned for providing the test results back to the appropriate recipient would be relatively low. However, if the test results are complicated and involve a response beyond the simple response, the fee apportioned for providing the test results is accordingly higher.

Consistent with embodiments of the present invention, methods and systems for managing a schedule of remote tests are disclosed. The methods and systems allow for a centrally managed schedule of tests to be established. In this way, the impetus for tracking and prompting testing events is placed with a central server, rather than a test subject. For example, a central server may provide reminders to a test subject before a scheduled testing event or after the subject has failed to complete a scheduled testing event.

In accordance with embodiments of the invention, a subject generally provides a sample, which is input to a test kit. The sample may comprise a bodily substance, such as blood, urine, fecal matter, or saliva, for example. The test kit then produces data relating to the sample and transmits the data to a central server. The central server analyzes the data to provide results. The central server may archive the data, transmit results, and bill a party for a fee related to the test. The focus of this application is on the aspect of how such centrally-analyzed data can be used for remote testing a subject according to a centrally managed schedule of tests.

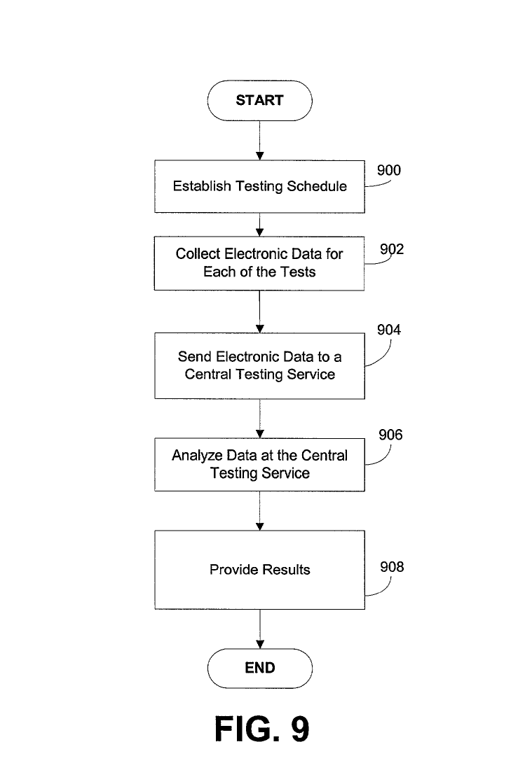

In the context of the above described testing kit 100 and data service 200, an exemplary method in accordance with an embodiment of the present invention is illustrated in FIG. 9. Turning now to stage 900 in FIG. 9, a testing schedule is established that typically comprises one or more pre-selected tests, each performed on a subject. In general, the schedule is a listing of one or more tests that are associated with the subject. The tests on the schedule may be of the same type, but prescribed to occur on different days and/or times. Alternatively, the tests may be completely different types of tests requiring a wholly different type of cartridge and sensor array.

Typically, the tests are established for the subject by a third party, such a medical provider, scientist, regulatory agency, or other entity, depending upon historical data related to the subject. In a medical example, the tests may be part of a clinical protocol of tests (i.e., specific tests prescribed as part of a treatment or study) or series of monitoring tests after a drug or chemical therapy has been applied to the subject patient. A physician may review the subject patient's historical medical records and establish a series of tests to be performed on the subject. The series of tests is listed in the schedule, which can be advantageously and centrally managed by the central data service. Alternatively, in a water testing example, the schedule of tests may include testing for different types of bacteria or waste contaminants within a body of water over a period of time and at different times of the day. The precise times and days for testing may be established based on historical data for the body of water and/or historical data regarding the surrounding environment (e.g., operational times for neighboring industry, etc.).

In the exemplary embodiment, the schedule of tests is implemented as a test schedule file 250 on data service 200. Typically, this test schedule file 250 is uploaded into the data service 200 from third party systems 050, such as doctors, hospitals, pharmaceutical companies, clinical laboratories, insurance companies, regulatory agencies, and the like. However, the test schedule file 250 may be created directly on data service 200 without uploading. Once within the data service 200, the test schedule file 250 may be kept stored in non-volatile memory or may be kept in volatile memory.

Using cartridge reader 130, electronic test data are collected for each of the one or more tests at stage 902. Based on the above description of the cartridge reader 130 and cartridge 110, those skilled in the art will appreciate that each of the one or more tests may require the acquisition of a sample 115. Once electronic data are collected for a given test, they are sent to the data service 200, as shown at stage 904.

At stage 906, the data service 200 analyzes the electronic data. The results of this analysis may then be provided to the subject or any other party, as illustrated at stage 908, and are stored within analysis results data database 243. Storing such test results is typically accomplished by creating a record in the database associated with the subject and the particular test performed from the scheduled tests. In the exemplary embodiment, an entry in the test schedule file 250 may also be updated or, more generally, flagged after the analysis of electronic data for the particular test. Such a flagging operation is indicative that the particular test in the schedule is complete. Those tests not flagged are deemed to be incomplete, further indicating that the schedule is still incomplete.

Data service 200 periodically reviews test schedule file 250 to identify if any of the tests within the schedule have been missed. This is normally accomplished by data analysis module 220, which analyzes the tests not flagged within the file and the prescribed dates for each outstanding tests' completion. If one of the tests is identified as having been missed, data service 200 typically issues a reminder to the subject so that the scheduled tests may be completed according to the schedule.

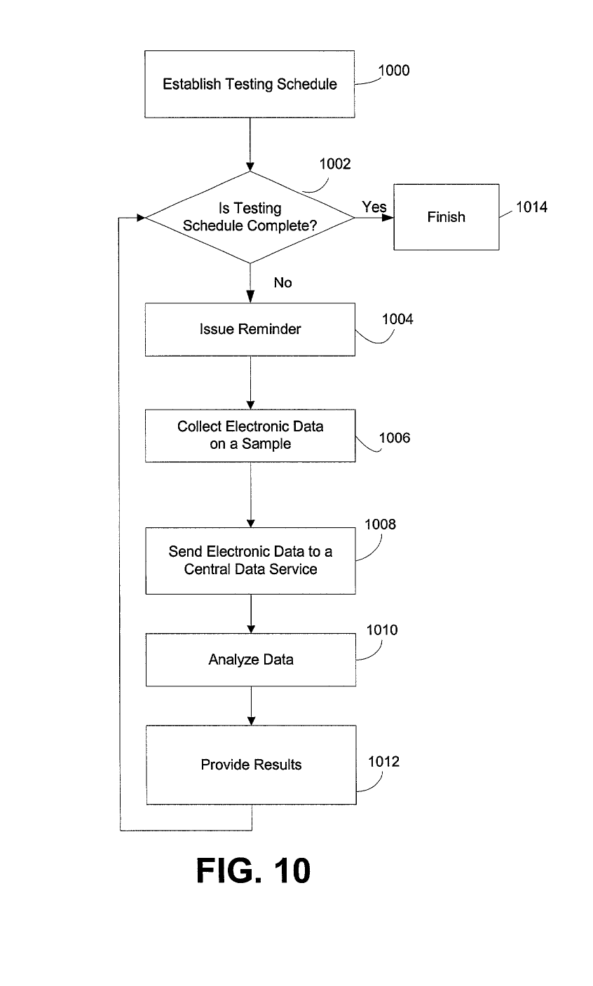

Referring now to FIG. 10, an exemplary method incorporating a reminder function will now be described in conjunction with the testing schedule for the remote testing device. In stage 1000, a testing schedule is established having one or more tests. At stage 1002, a review of the testing schedule is performed and a determination is made as to whether the testing schedule is complete. In the exemplary embodiment, this is accomplished by reviewing the contents of test schedule file 250.

If stage 1002 is answered in the negative, a reminder is issued at stage 1004. In general, a “reminder” should be broadly interpreted to mean any type of notification related to the testing schedule. The reminder may be issued using any communication format, such as an electronic mail message, a piece of mail, a telephone call, a facsimile, a page for receipt by a pager, or any other suitable communication. Moreover, an indicator on cartridge reader 130 may provide the reminder, for example, by illumination of an indicator light or by providing a message on a Liquid Crystal Display (LCD) or a monitor.

In response to the reminder and subsequent use of cartridge reader 130, electronic test data are collected for each of the one or more tests at stage 1006. Each of the one or more tests may require the acquisition of a sample 115. Once electronic data are collected, they are sent to a data service 200, as shown at stage 1008. At stage 1010, the data service 200 analyzes the data. Results may then be provided to the subject or any other party, as illustrated at stage 1012. The method then revisits the determination at stage 1002 and is repeated until the testing schedule is completed. Once stage 1002 yields an affirmative determination, the method is finished, as illustrated at stage 1014.

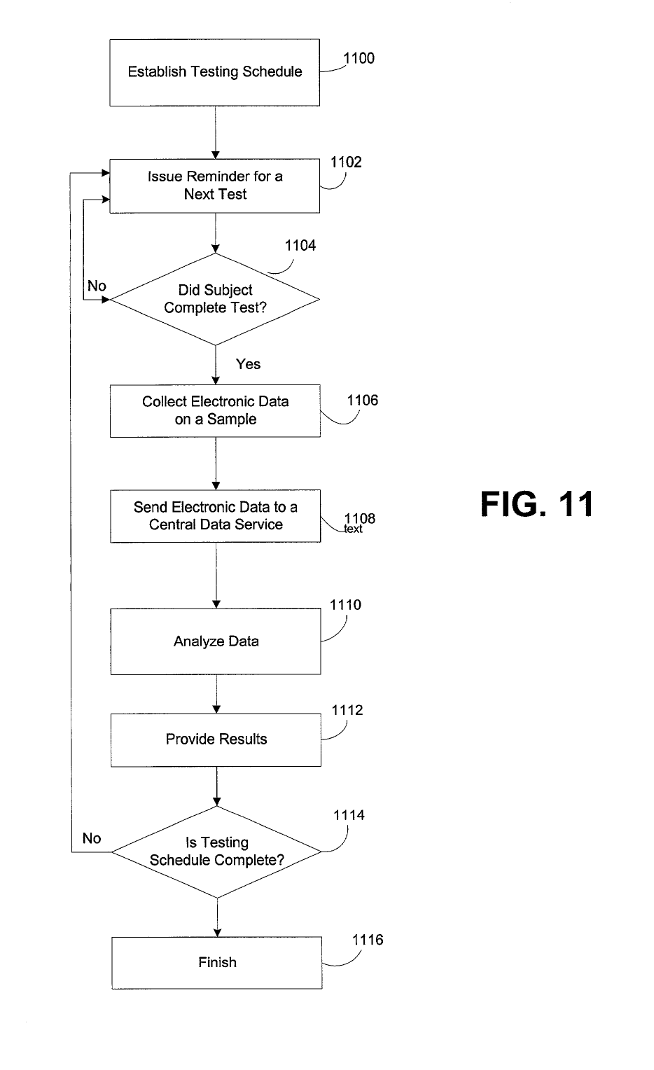

FIG. 11 is a flowchart of another exemplary method for completing a testing schedule with a reminder function. At stage 1100, a testing schedule is established, and, at stage 1102, a reminder is issued for a next test. As mentioned previously, the reminder may take many forms, including by example an electronic mail message, a piece of mail, a telephone call, a facsimile, a page, or any other suitable reminder. In stage 1104, a determination is made whether a subject completed the next test. When the determination yields a negative result, the reminder stage 1102 is repeated until an affirmative result is obtained. Reminder stage 1102 may be repeated at regular intervals, with varying frequency, with increasing frequency, or substantially constantly until the next test is completed.

Once the next test is completed, collection stage 1106, sending stage 1108, analysis stage 1110, and providing stage 1112 occur in a similar manner to stages 1006, 1008, 1010, and 1012 of FIG. 10, respectively. At stage 1114 of FIG. 11, a determination is made whether the testing schedule established at stage 1100 is complete. An affirmative determination yields the finish of the method, as shown in stage 1116. If the testing schedule is not complete, a reminder is issued for a next test in the schedule, as shown at stage 1102.

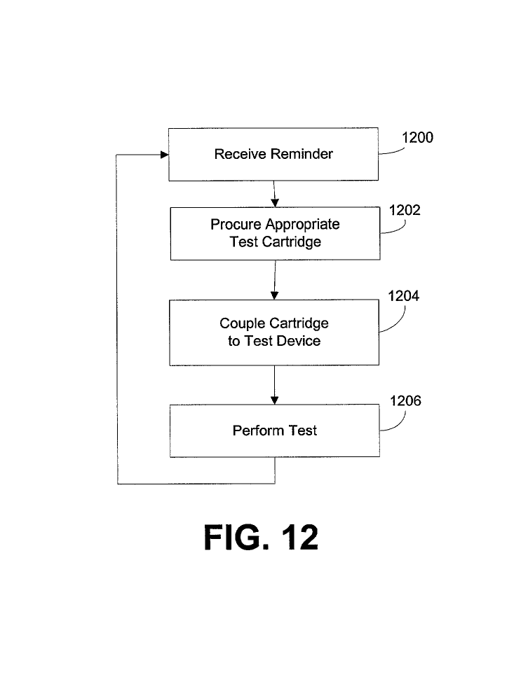

FIG. 12 illustrates an exemplary method according to the present invention from the perspective of a test subject. At stage 1200, the subject receives a reminder, typically from data service 200. The reminder may indicate a type of test which should next be performed and a time at which the test should be performed. Typically, the scheduled time for an upcoming test is listed in the testing schedule. The reminder may also repeat until the desired test is performed.

Upon receiving the reminder, the subject then procures a test cartridge 110 appropriate for a next test (Stage 1202). The subject then couples the appropriate test cartridge 110 to a cartridge reader 130, as shown at stage 1204. In stage 1206, the appropriate test is performed. The method of FIG. 12 may optionally loop until no more reminders are received, thus indicating the end of a testing schedule.

Consistent with embodiments of the present invention, methods and systems for screening a subject as a potential participant in a study or clinical trial are disclosed. The methods and systems allow a subject, such as a medical patient, to submit to one or more remote screening tests without being required to visit a clinic or laboratory where samples from the subject are typically submitted to technicians. In this way, a central server may use a set of preliminary results to determine a subject's suitability for inclusion in a clinical trial. Test subjects may be specifically solicited for inclusion in a clinical trial based on a pre-clinical trial screening, or they may be culled, via the central server, from a pool of test subjects coincidentally or historically undergoing similar screening tests. In other words, the use of a central data service as part of remote testing enables a targeted search for subjects meeting certain clinical or test result requirements. Permission for inclusion should be solicited from the subject, wherein the privacy and anonymity of the subject would be respected until permission is actually granted. Given the advantages of such central server-based targeting of participants, billing to the third party can be structured in stages. A first billing event may occur when a candidate participant is initially discovered with an addition billing event occurring when the candidate actually registers to be a participant in the study or clinical trial.

In accordance with embodiments of the invention, a subject generally provides a sample, which is input to a test kit. The sample may comprise a bodily substance, such as blood, urine, fecal matter, or saliva, for example. The test kit then produces data relating to the sample and transmits the data to a central server. The central server analyzes the data to provide results. The central server may then determine the subject's suitability for inclusion in a clinical trial. The central server may then solicit the consent of the subject to participate in the clinical trial. Billing functions are provided both after finding a potential participant and after obtaining the potential participant's consent. The focus of this application is on the aspect of how centrally analyzed data can be advantageously used for screening a subject as a potential participant for a study or clinical trial.

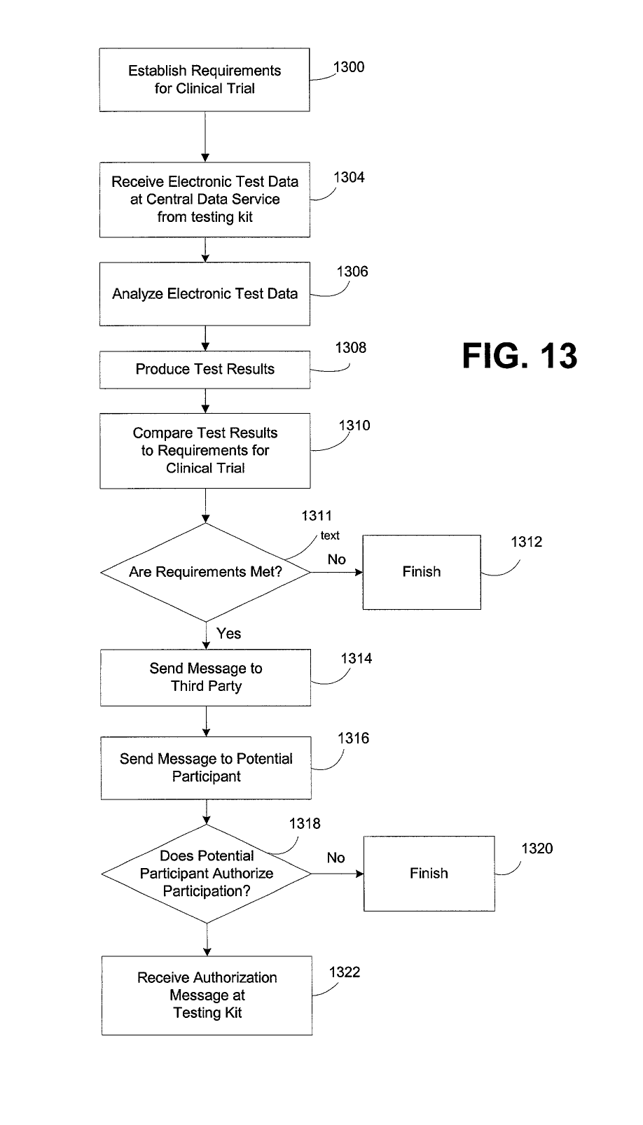

In the context of the above description, an exemplary method of screening a subject for participation in a clinical trial consistent with the present invention will now be described with reference to FIG. 13. In stage 1300, requirements are established for a clinical trial. In general, a clinical trial should be broadly interpreted to mean any experimental trial or study where a third party tester is looking for one or more reactions from the participants in response to stimuli. For example, a pharmaceutical company or medical researching facility may conduct a clinical trial using participants that have contracted the HIV virus. These HIV virus participants are subjected to various stimuli, such as a new drug therapy. Typically, the clinical trial is conducted so that part of the participant population unknowingly acts as a control while the other part of the population takes the new drugs. Periodic testing is normally part of such studies or clinical trials and may be used to monitor the population as the trial proceeds. Another example of a clinical trial may be a study of the effect of a pesticide on a particular type of corn.

The requirements for participation in such a clinical trial may be very simple (such as growing the particular type of corn) or may involve meeting a complex medical profile with a desired medical history and current medical conditions. For example, the requirements may comprise blood type, antibody presence, virus presence, and the like. Once the requirements for the clinical trial are established, data service 200 can review any test results to see if they meet one or more of the requirements established at stage 1300.

A subject would then provide a sample to cartridge reader 130, which then would send electronic test data for central testing, analysis, interpretation and further processing as discussed below. In stage 1304, the electronic test data are received from cartridge reader 130 at data service 200. These electronic test data are then analyzed according to an appropriate test at stage 1306, and test results are produced at stage 1308. In one embodiment, the appropriate test can be any test that involves data service 200. However, another embodiment contemplates that the appropriate test is a test that is directed specifically to find results that can meet one or more of the clinical trial requirements.

The test results produced in stage 1308 are then compared against the requirements for the clinical trial at stage 1310. In the exemplary embodiment, data service 200 is programmed to operate as a background monitoring system that compares all test results as they are produced to a set of requirements for each clinical trial under consideration. This would be accomplished using a daemon (not shown) or other type of software process as part of data analysis module 220. That portion of data analysis module 220 reviews test results as they are produced and compares them to a listing of requirements from a test requirement file (not shown) that define a desired test participant profile.

A determination is made at stage 1311 whether the requirements are met. More particularly stated, a determination is made whether a match exists between all or a subset of the test results and one or more requirements that collectively form the desired test participant profile. If they are not, the method is finished at stage 1312. If stage 1311 yields an affirmative determination, a message is sent to a third party at stage 1314. In the exemplary embodiment, the message of stage 1314 would notify the third party of the existence of a potential participant who meets the requirements of the clinical trial. Optionally, a finder's fee or other type of fee is billed to the third party. This finder fee becomes the third party's payment at a typically nominal level for even finding a potential participant for the third party's clinical trial. For example, if a pharmaceutical company is seeking patients with AB type blood and a history of heart disease, data service 200 is operative to provide an indication that a match exists for the company's clinical trial requirements without having to name the subject's name. At this point, the company would be billed or would have their billing records flagged for later production of a bill. Flagging of billing records involves a fee, generally referred to as a finder fee and which is typically a nominal fee.

At stage 1316, a message may also be sent to the potential participant. Typically, a potential participant may be notified that he or she meets the requirements for the clinical test. Information regarding the clinical trial may also be provided to the subject who is the potential participant. The message may optionally ask for the authorization of the potential participant to participate in the clinical trial. Additionally, the message may include enrollment information or documents for participation in the clinical trial.

If the potential participant authorizes his or her participation in the clinical trial at stage 1318, the method proceeds to stage 1322 where an authorization message is received at data service 200, as shown in stage 1322. Further, the third party may be billed for an additional fee (also called a participation bonus) at this stage for finding a participant that desires to participate in the clinical trial. However, if no authorization is granted, the method finishes at stage 1320.

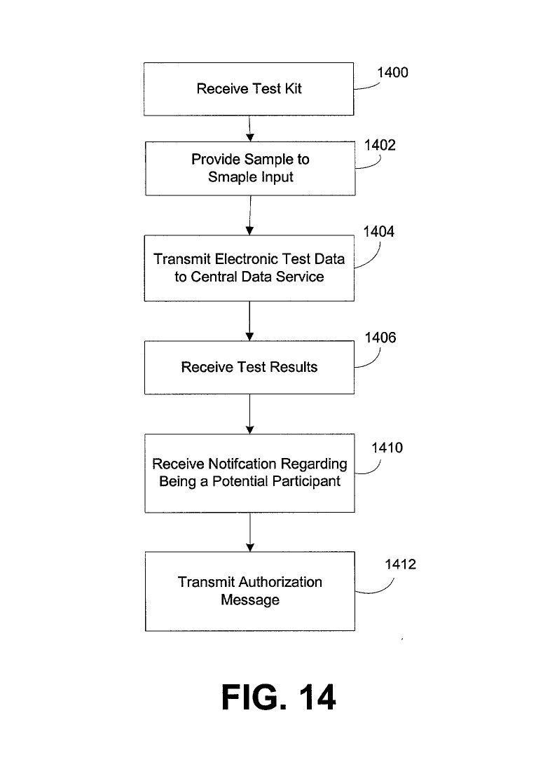

FIG. 14 illustrates the exemplary method of screening a subject for participation in a clinical trial from the perspective of a subject/potential clinical trial participant. At stage 1400, the subject receives a test kit by mail, courier, or other suitable delivery means. In an exemplary embodiment, the test kit comprises a cartridge reader 130 and a cartridge 110 tailored to the desired test parameters. The subject then provides a sample input on cartridge 110, as illustrated at stage 1402. At stage 1404, electronic test data related to the sample are then transmitted to data service 200. Test results are received at cartridge reader 130 at stage 1406, wherein the test results may comprise raw data or an interpretation of the electronic test data. At stage 1410, notification is received at cartridge reader 130 regarding the subject's suitability as a potential participant in the clinical trial. The notification of stage 1410 may also comprise a request for authorization to include the subject in the clinical trial. Optionally, at stage 1412, the subject may transmit an authorization message to data service 200, thus indicating his or her desire to participate in the clinical trial.

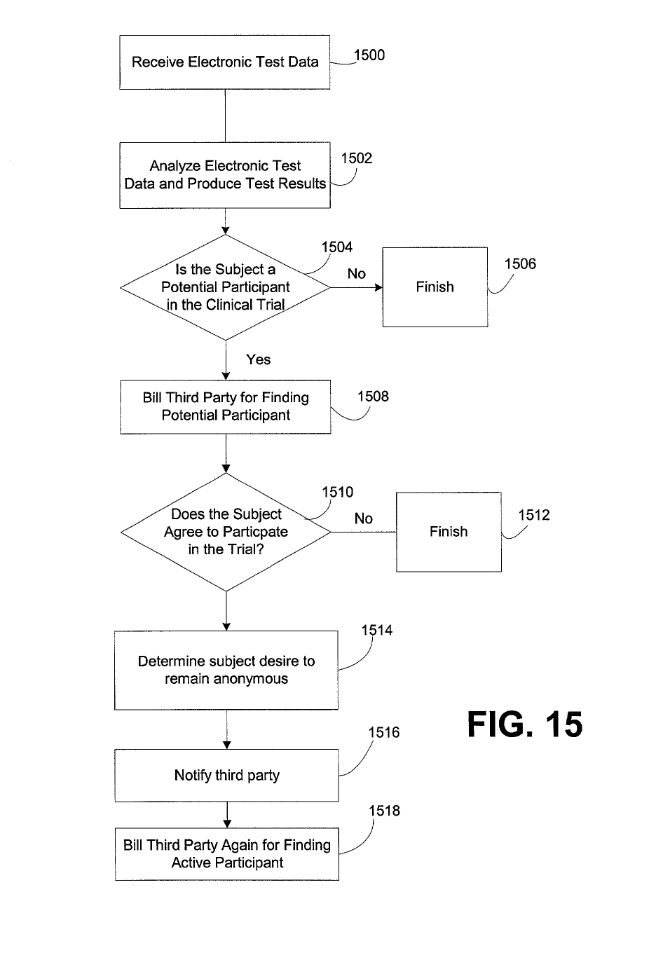

An illustration of a method of billing related to the aforementioned screening methods is provided in FIG. 15. At stage 1500, electronic test data received at data service 200. These electronic test data are analyzed to produce test results, as shown at stage 1502. A determination is then made at stage 1504 whether the subject is a potential participant in the clinical trial. This determination would be based on, for example, whether the subject meets the requirements of the clinical trial. If the subject is not a potential participant, the method is finished at stage 1506. However, if the subject is a potential participant, a third party is billed at stage 1508 by billing service 040 for finding a potential test participant. In the exemplary embodiment, the amount of that the third party is billed at this point is designated a finder fee, typically at a nominal level (as opposed to a higher participation bonus fee). Those skilled in the art will appreciate that the third party may comprise, for example, a university, a researcher, a corporation, or any other entity desirous of finding participants for a clinical trial.

In stage 1510, it is determined whether the subject, who is a potential participant, actually agrees to participate in the clinical trial. This determination may be based on some input by the subject, such as an electronic message or other indication from the subject of his or her desire to participate. If stage 1510 results in a negative determination, the method finishes at stage 1512. However, if stage 1510 results in a positive determination, the third party is billed again for finding a potential participant who meets the requirements of the clinical trial and who is willing to participate in the clinical trial at stage 1514. Typically, the third party's bill will include a participation bonus that is substantially larger than the finder fee billed at stage 1508. Such billing amounts will vary according to the relative complexity of the test participant profile and other market force factors (e.g., competition, etc.).

Reference will now be made to another embodiment according to the present invention, where the embodiment is a method for screening an isolated or quarantined test subject using a remote testing device. This embodiment allows a quarantine or isolation subject to submit to one or more screening tests using a remote testing device. In this way, the ongoing necessity for the quarantine or isolation can be centrally and efficiently determined. Furthermore, the remote testing device facilitates quicker test results, so that quarantines need not be unnecessarily prolonged. Similarly, the remote testing device may be readily deployed to a place of quarantine or isolation, thus obviating spatial limitations associated with a quarantine subject. Test data from the remote testing device may then be processed at a central server, wherein clearances from the quarantine or isolation may be granted based on test results from the central server.

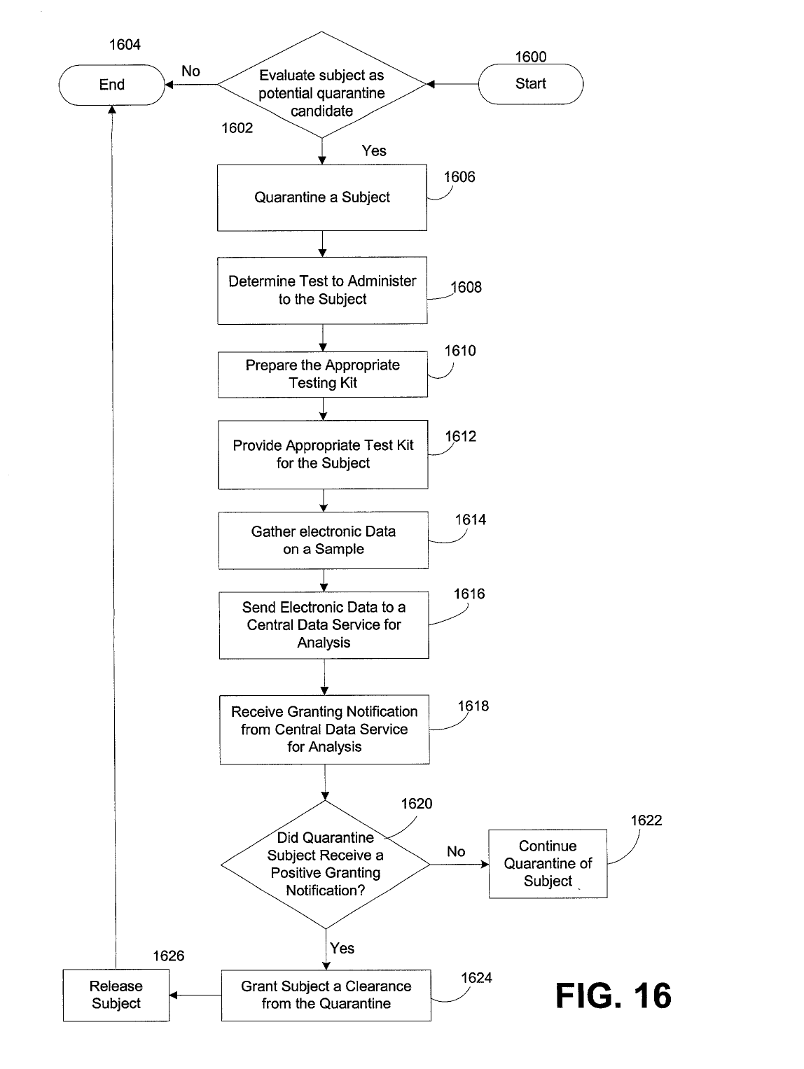

In the context of the above description regarding sampling, gathering electronic data and centrally analyzing such data, an exemplary method for screening a quarantined or otherwise isolated test subject using a remote testing device is described as shown in FIG. 16 consistent with an embodiment of the present invention. With reference to FIG. 16, the process begins at stage 1600. At stage 1602, a subject is evaluated for being a potential candidate for isolation or for quarantine. If this evaluation is negative, the subject is released and the process ends at stage 1604. If is determined that the subject is in fact a candidate for quarantine or isolation, the process continues to stage 1606. The term “quarantine” should be broadly interpreted to mean an enforced isolation or restriction of free movement to prevent the spread of a health risk or threat, such as a contagious disease. In some instances, the term quarantine may be used when a subject is detained at a port of entry facility under enforced isolation to prevent disease from entering a country. Examples of such a quarantine may include a quarantine of a human or animal subject due to a suspected or actual disease, or a quarantine of an immigrating subject based on immigration laws, regulations, or requirements. Further still, an exemplary quarantine may involve isolating a source of drinking water or isolating a body of water from human contact to prevent the spread of disease or other contaminants in the water.

In stage 1608, a determination is made as to what test should be administered to the quarantine subject. In one embodiment, the test is related to the reason for the quarantine, and the test may be performed to validate, verify, avert, or obviate the reason for the quarantine. For example, if the quarantine is for inbound passengers from a third world country, the test may be determined according to a schedule of tests to be to detect the presence of hepatitis, cholera, or some other undesirable disease. In another example, if the quarantine is setup to isolate a body of water from human contact, the test may be to detect the presence of a particularly undesirable bacteria in the water.

Based on the determination of stage 1608, an appropriate test is prepared in stage 1610 and is provided for the subject in stage 1612. In the exemplary embodiment, the test kit comprises a cartridge reader 130 and a replaceable cartridge 110, wherein cartridge 110 is related to the desired test. Depending upon the test subject and the reasons for isolation or quarantine, the appropriate type of replaceable cartridge 110 may be included as part of the test kit. Those skilled in the art will appreciate that in some situations, the test kit is the cartridge without the test device.

Once an appropriate sample is obtained from the quarantine subject, electronic data are gathered on the sample at stage 1614. This may be accomplished by, for example, placing the sample in a sample input of cartridge 110 and activating cartridge reader 130. Typically, calibration indications are analyzed to ensure the cartridge reader 130 and cartridge 110 are functioning within acceptable parameters for the desired test. Once the proper functioning of cartridge reader 130 and cartridge 110 are found, electronic data are gathered by cartridge reader 130.

These electronic data are then transmitted from cartridge reader 130 to data service 200 for analysis, as shown at stage 1616. The data are analyzed at data service 200, thus producing test results. Based upon the test results, data service 200 issues a granting notification. In the exemplary embodiment, the granting notification is an electronic message from data service 200 to testing device 105.

The granting notification is received, typically at the testing device, as illustrated at stage 1618. A determination is made at stage 1620 if the quarantine subject received a positive granting notification. In the exemplary embodiment, data service 200 is operative to review the test results and make this determination based upon predetermined thresholds or acceptable ranges that are established for the particular test. A positive granting notification may comprise, for example, a message stating that the reason for the quarantine was obviated by the test results. If a positive granting notification is received, the subject may optionally be granted a clearance (Stage 716) and released from the quarantine (Stage 1626). If a negative granting notification is received, indicating that the reason supporting the quarantine remains, the quarantine of the subject is continued, as shown in stage 1622.

In another embodiment, the quarantine is setup to isolate a body of water from human contact. It is anticipated that the body of water may include but is not limited to be a lake, river, a part of the ocean, a man-made body of water, or simply a water supply with one or more access points. Essentially, the quarantine is a forced isolation of the body of water. This may be implemented as an order from municipal authorities advising and effectively isolating consumers from a drinking supply due to potential dangerous contamination of the supply. For example, there may be a chemical spill in a water treatment plant. Alternatively, there may be an undesirable amount of bacteria in the body of water, such as when a “red tide” is discovered in a part of the ocean or other saltwater body of water. In either case, the body of water may not be fit for contact with humans or animals without sufficient testing and/or treatment of the water.

Samples of the test subject, e.g., the body of water, can advantageously be gathered from multiple locations or access points. Using multiple remote testing devices, such as cartridge reader 130, a team of testing technicians can rapidly deploy to process the samples, upload their results to a central server, such as data service 200, and determine if the subject body of water remains a threat. When the body of water is a drinking water supply, each of the test devices can sample water coming from different access points (e.g., tap water from houses in different neighborhoods, water in different parts of a sewage treatment plant, water at different distribution points along a suspect water pipe, etc.).

The threat analysis is typically performed as a determination for a particular bacteria or other unwanted or undesirable component of the water. In other words, if any sample yields test results that are above a certain threshold, the subject body of water is deemed to require additional isolation or quarantine time.

It is further contemplated that the threat analysis may be implemented as a comparison between profiles of test results. The test results (e.g., pH, bacteria level, etc.) may yield different results for each of the samples, but collectively the results identify a particular testing profile for the subject body of water. Under normal or acceptable conditions, the testing profile may have or be in acceptable ranges depending on the type of tests performed and the exact threshold of acceptability. However, comparing the current profile of test results to those when the subject is in an acceptable condition may determine if the subject body of water is considered a threat.

In another example, water may flow through a distribution pipe that has an intake at a particular location on a lake. This water may be a source of drinking water for a community. Measurements taken using multiple test devices on water from several shoreline locations around the lake and from several underwater locations near the intake help to establish a profile of test results. If one of more of the test results falls out of a predetermined range (such as a range for bacterial concentration or mercury levels) for that location or access point, the collective profile of test results indicates that the subject body of water should remain quarantined or isolated. In other words, the subject water should be isolated from human contact due to the remaining threat of contamination or disease.

Yet another embodiment of the present invention involves the determination of food safety. By way of example, the sample preparation 020 shown in FIG. 2 may comprise several steps. The device that implements this preparation can be housed in one system that may be automatically interfaced into the subject 010, or alternatively it may require manual steps from the testing kit 100 operator. The original sample from the subject 010 is first pre-processed. This pre-processing may include grinding, drying, milling, sonication, or other processes. This results in a first intermediate product which is combined with reagents (such as acids, enzymes, lysing agents, nucleaic acids, etc.). The first intermediate and combined reagents may be processed, for example by alternately heating and cooling the combination, or sonicating. These reagents are used to extract, purify, concentrate, or remove components of the first intermediate product. The result is a second intermediate product. Post-processing steps which may include filtering, centrifuging, and incubating are used to prepare the test substance. The resulting material represents the sample 115 and will contain all of the components of interest from the original item (such as E. Coli 0157:H7). It may also be a small portion of the products of the post-processing steps with excess material discarded as waste.

As described in previous embodiments, the sample 115, containing the target amplicon(s) is then introduced to the cartridge 110 and electronic information is then generated by the cartridge reader 130 and transmitted to a data service 200. In this particular embodiment, the data service 200 is a data manipulation system. This process will vary depending on the specific test being performed and the environment within which the test is performed.

The electronic information transmitted to the data manipulation system is processed through appropriate Quality Control functions. The Test Station along with a QC process is monitored to ensure compliance with Quality Assurance standards. The results can then be transmitted from the data manipulation system databases to any other database such as, for example, for tracking and isolation of the source for the detected contamination.

These results can then be augmented with information contained is these databases such as the conditions that were used to raise the food animal being tested. These results can then be transmitted to consumers such as NPPC or other Associations, Food Producers, USDA or EPA, Food services such as McDonalds, Food retailers such as Wal Mart, etc.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for processing electronic information associated with predetermined characteristics of a biological subject, the method comprising:
providing a testing kit to a location proximate the biological subject;
obtaining a representative sample of the biological subject;
introducing the sample to the testing kit;
the testing kit performing a pre-selected test or series of tests;
the testing kit generating electronic information representative of at least one characteristic of the sample;
transmitting the electronic information to a data service that is functionally not proximate the testing kit;
receiving the electronic information at the data service;
the data service processing the electronic information to determine a test result representative of the at least one sample characteristic of the sample;
the data service generating and electronically transmitting the test result to at least one of a testing kit operator, the subject, a provider, and a third party; and
the at least one of the testing kit operator, the subject, the provider, and the third party receiving the test result.

2. The method of claim 1, wherein the electronic information includes descriptive information associated with the at least one of the testing kit operator, the subject, the testing kit, the testing kit location.

3. The method of claim 2, further comprising the testing kit:
generating the electronic information representative of at least one of an acceptable operation of the testing kit and an acceptable introduction of the sample to the testing kit; and
transmitting the electronic information to the data service correlated with the electronic data resulting from the testing.

4. The method of claim 3, wherein determining a test result further comprises analysis of the electronic information to determine at least one of the acceptable operation of the testing kit and the proper introduction of the sample to the testing kit.

5. The method of claim 4, further comprising reporting by the data service to at least one of the subject, the provider and the third party of the results of the electronic information representing at least one of the acceptable operation of the testing kit and of the proper introduction of the sample.

6. The method of claim 2, further comprising communicating testing kit identification information and subject identification information to the data service.

7. The method of claim 6, further comprising associating a testing kit identification with a subject record.

8. The method of claim 2, further comprising communicating testing kit identification information to the data service.

9. The method of claim 2, wherein the testing kit comprises a cartridge for receiving the sample therein, the method comprising:
determining an in-service date associated with the cartridge;
accessing a second set of electronic information comprising expiration information for the cartridge;
determining an expiration status of the cartridge; and
transmitting a notification to at least one of the subject, the testing kit operator, the provider, and the third party as to the status.

10. The method of claim 8, wherein the testing kit comprises a cartridge for receiving the sample therein, the method further comprising:
determining an in-service date associated with the cartridge;
accessing a second set of electronic information comprising expiration information for the cartridge;
determining the expiration status of the cartridge; and
transmitting a notification to at least one of the subject, the testing kit operator, the provider and the third party as to the status.

11. The method of claim 2, wherein the descriptive information associated with the testing kit location is information obtained from a Global Positioning Satellite system.

12. The method of claim 11, wherein equipment for receiving and accessing the Global Positioning Satellite system location information is integral to a reader operable with the testing kit.

13. The method of claim 1, further comprising confirming the test results by one of reusing the testing kit and by using another testing kit.

14. The method of claim 1, further comprising storing the test results such that the results can be retrieved or reviewed at a later time.

15. The method of claim 1, further comprising notifying at least one of the subject and the testing kit operator that the results were transmitted to at least one of the provider and the third party.

16. The method of claim 1, further comprising indicating to at least one of the subject, the provider and the third party that the testing kit performed within allowable accuracy limits.

17. The method of claim 16, wherein the indicating is accomplished within a timeframe so as to avoid delay in providing a conclusion.

18. The method of claim 1, further comprising encrypting of the electronic information prior to transmitting to the data service.

19. The method of claim 18, further comprising decrypting by the data service of the received electronic information.

20. The method of claim 1, further comprising preparing the sample prior to introducing the prepared sample to the testing kit.

21. The method of claim 20, wherein the preparing includes one or more of liquification, addition of reagents, temperature treatment, physical treatment and chemical treatment.

22. The method of claim 1, wherein the test results are available within approximately an hour of the receiving of the electronic information by the data service.

23. The method of claim 1, wherein the testing kit contains at least one of a disposable sample cartridge, a re-useable sample collection cartridge, and a sample transport cartridge.

24. The method of claim 23, wherein the testing kit further includes at least one cartridge reader to be used in conjunction with the cartridge for performing a desired test.

25. The method of claim 1, further comprising storing of the electronic information received by the data service such that it may be used at a later time.

26. The method of claim 1, further comprising evaluating the test results to determine at least one of further testing and treatment requirements.

27. The method of claim 1, further comprising minimizing potential sample contamination through a reduction of at least one of the provider and the third party handling of the sample from the time of collection to the time of introduction to the testing kit.

28. The method of claim 1, where the location of the sample is remote from at least one of the location of a provider and the location of the third party.

29. The method of claim 1, wherein the location of the sample is proximate at least one of the location of the provider and the location of the third party.

30. The method of claim 1, wherein the sample comprises saliva.

31. The method of claim 1, further comprising at least one of allocating a fee and authorizing a disbursement for accomplishing at least a portion of the method for processing the electronic information.

32. The method of claim 31, wherein at least one of the fee allocating and disbursement authorization is initiated by a billing transaction when the electronic information is received at the data service.

33. The method of claim 31, wherein at least one of the fee allocating and disbursement authorization is initiated by a billing transaction from the data service when electronically transmitting test results to at least one of the provider and the third party.

34. The method of claim 31, further comprising selling the testing kit.

35. The method of claim 34, wherein the selling is associated with the cost of the determining the test results by the data service.

36. The method of claim 34, wherein the selling is associated with at least one of the cost of the storage of the electronic information and the determining of test results by the data service.

37. The method of claim 34, wherein the selling is associated with at least one of the cost of transporting the testing kit to the sample location and the amount pre-determined for disbursement to at least one of the provider and third party.

38. The method of claim 34, wherein the selling is associated with the cost of transmitting the test results to at least one of the subject, the provider, and the third party.

39. The method of claim 31, further comprising at the data service, communicating billing information associated with the testing to a compensator.

40. The method of claim 1, further comprising accomplishing all required laboratory certification activities at the data service location.

41. The method of claim 40, wherein the proximate location and the data service are operated such to avoid requirements of CLIA certification at proximate locations and wherein CLIA requirements are met at the data service.

42. The method of claim 1, further comprising:
the testing kit electronically transmitting a test request to the data service;
the data service receiving the test request;
based on the information contained in the test request, the data service accessing existing electronic information and retrieving a pre-defined operation required to perform the requested test for the testing kit transmitting the test request;
the data service generating controlling information representative of the pre-defined operation;
the data service transmitting the controlling information to the testing kit;
the testing kit receiving the controlling information and determining the pre-defined operation; and
the testing kit performing the pre-defined operation.

43. The method of claim 42, wherein the test request is initiated by at least one of the subject, the provider, and the third party located proximate the testing kit.

44. The method of claim 42, wherein the test request is initiated by at least one of the provider and the third party remote from the testing kit.

45. The method of claim 42, wherein the pre-defined operation and the resulting controlling information is under functionally centralized control of at least one individual.

46. The method of claim 1, wherein the biological subject is a human.

47. The method of claim 46, further comprising:
the introducing of the testing kit to the subject at subject initiation;
the receiving of a subject-initiated request for the testing kit to determine at least one subject characteristic by at least one of the provider and the third party;
the providing of the testing kit by at least one of the provider and the third party;
the obtaining of a small amount of the sample from the subject without participation by a licensed laboratory technician;
the interpreting of the received electronic information at the remote diagnostic data service;
the determining of the test result based upon an interpreting of the electronic information; and
the making of the test result available to at least one of the subject and the provider for subject consultation.

48. The method of claim 47, further comprising after receiving the test result, introducing at least one of a drug, medication, and treatment to the subject based at least in part on the test result.

49. The method of claim 1, wherein the biological subject is an animal.

50. The method of claim 49, wherein the animal is at least one animal used for at least one of developing drugs, evaluating drugs, evaluating medications, and evaluating treatments;
the sample is such that the taking of the sample does not jeopardize the animal's life or otherwise preclude the taking of samples from the animal; and
the introducing of the sample to the testing kit is performed to a pre-determined schedule.

51. The method of claim 50, further comprising:
interpreting the received information for generating a test result;
electronically storing the test result at the data service; and
correlating the test result to support the evaluating, and developing of a drug, medications, and treatment.

52. The method of claim 1, wherein the biological subject is at least one of one of a plant, a collection of plants, and parts thereof.

53. The method of claim 1, wherein the biological subject is at least one of an environmental ecosystem, and elements thereof.

54. The method of claim 1, further comprising adding at least one of subject biometric, biographical, and demographic data to the electronic information transmitted to the data service.

55. The method of claim 54, further comprising:
the data service incorporating the at least one of the biometric, biographical, and demographic information with the test results;
the data service transmitting the at least one of the biometric, biographical, and demographic information to the subject, a provider or third party along with the test results; and
the subject, a provider or third party verifying that the reported results represent the intended sample from the correct subject.

56. The method of claim 55, wherein said verifying is made by the testing kit at the location of at least one of the subject, the provider, and the third party.

57. The method of claim 1, further comprising:
the provider determining a set of subject characteristics to be determined prior to a scheduled meeting between the subject and a provider;
the provider or a third party defining a testing kit based on the set of characteristics;
the provider or a third party preparing the defined testing kit;
at least one of the provider, and the third party including at least one disposable, reusable sample collection, and sample transport cartridge in the testing kit;
at least one of the provider and the third party introducing the testing kit to the location of the subject and the subject location being other than that of the provider;
at least one of the subject and the testing kit operator introducing the sample to the at least one disposable, reusable sample collection, and transport device;
the subject or testing kit operator introducing the at least one of the disposable and reusable sample collection and sample transport cartridge to a cartridge reader;
the cartridge reader generating electronic information representative of sample characteristics as determined by the cartridge reader;
the cartridge reader generating and transmitting the electronic information to the data service;
the data service interpreting the electronic information to determine a test result representative of the subject characteristics of interest; and
the data service electronically transmitting the test result to at least one of the subject, the provider, and the third party.

58. The method of claim 57, further comprising the data service reporting to at least one of the provider and the third party that the electronic information was received by the data service.

59. The method of claim 57 wherein the determining a set of subject characteristics comprises;
receiving a request for the history of the subject;
accessing at least one record in a database, the at least one record being related to the subject; and
providing the contents of the at least one record to the third party.

60. The method of claim 57, further comprising at the data service, performing an analysis to determine a recommendation for additional testing.

61. The method of claim 60, further comprising at the data service, the performing an analysis to determine a recommendation for additional testing includes comparing present data with prior data for the subject.

62. The method of claim 57, wherein the defined test kit provides for the testing for an allergic or interaction to a particular substance.

63. The method of claim 62, further comprising prior to obtaining the sample by a pre-defined time, introducing a screening-effective quantity of the substance to the subject.

64. The method of claim 57, wherein the testing kit provides for the testing for at least one of a response to a drug and medication based on changing conditions of the subject.

65. The method of claim 1, further comprising using biometrics to associate the sample with the subject.

66. The method of claim 65, wherein the receiving of the electronic information by the data service further comprises verifying the identity of an individual initiating the electronic information transmittal.

67. The method of claim 66, wherein the data service has access to information indicating authorization for at least one individual to transmit the electronic information.

68. The method of claim 66, wherein identification is based upon biometric data transmitted with the electronic information and representative of the individual initiating the transmittal of the electronic information.

69. The method of claim 68, further comprising the generation and transmittal of a notification to the individual initiating the transmittal of the electronic information of the resulting authorization determination.

70. The method of claim 69, further comprising providing a message by the data service to the individual initiating the transmittal of electronic information containing instructions for repeating the transmittal process if authorization is not initially successful.

71. The method of claim 1, further comprising encrypting of the test result by the data service before transmitting to at least one of the subject, the provider, and the third party.

72. The method of claim 71, further comprising decrypting of the transmitted test result at the location of at least one of the subject, the provider, and the third party.

73. The method of claim 72, further comprising determining an authorization of at least one of the subject, the provider, and the third party receiving the encrypted interpreting results to have access to the decrypted test result.

74. The method of claim 73, further comprising preventing decrypting of the received test result if the authorization determination is that the initiating individual does not have approved access to the decrypted test result.

75. The method of claim 1, further comprising correlating the electronic information or test result with at least one of a second set of electronic information, and the test results to generate correlation information.

76. The method of claim 75, wherein the electronic information or test results comprises electronic information about other samples from other subjects.

77. The method of claim 75, wherein the electronic information or test results comprises electronic information from other samples from the same subject.

78. The method of claim 75, wherein the electronic information comprises pre-defined test result standards.

79. The method of claim 78, wherein the correlation information is used for recommending further testing for subject.

80. The method of claim 78, further comprising:
the testing kit electronically transmitting a test request to the data service;
the data service receiving the test request;
based on the information contained in the test request, the data service accessing existing electronic information and retrieving the pre-defined operation or series of operations required to perform the requested test for the testing kit transmitting the test request;
the data service generating controlling information representative of the pre-defined operation or series of operations;
the data service transmitting the controlling information to the testing kit;
the testing kit receiving the controlling information and from it determining the pre-defined operation or series of operations; and
the testing kit performing at least one of the pre-defined operation and the series of operations.

81. The method of claim 80, wherein the test request is initiated by at least one of the subject, the provider, and the third party located proximate the testing kit.

82. The method of claim 80, wherein the test request is initiated by at least one of the provider and the third party while being remotely located from the testing kit.

83. The method of claim 75, further comprising analyzing the correlation information to determine if the test results lie within a pre-determined range of acceptable values.

84. The method of claim 83, further comprising notifying at least one of the subject, the provider, and the third party if the test results lie within a pre-determined range of acceptable values.

85. The method of claim 75, further comprising an electronic transmitting of the correlation information generated to at least one of the subject, the provider, and the third party.

86. The method of claim 75, wherein the correlating comprises comparing at least one of the electronic information and the test results with a second set of electronic information.

87. The method of claim 86, wherein the electronic information comprises a treatment database.

88. The method of claim 87, further comprising presenting treatment options data based on the treatment database and at least one of the test results of the subject, the provider, and the third party.

89. The method of claim 86, wherein the electronic information comprises a set of pre-defined clinical trial recruitment criteria where resulting correlation information is used to determine potential candidates to participate in clinical trials of at least one of medications and treatment.

90. The method of claim 89, further comprising notifying at least one of the subject, the provider, and the third party that the test results indicate the subject to be a potential candidate for inclusion into at least one clinical trial conducted by a fourth party.

91. The method of claim 90, further comprising transmitting at least one of an acceptance and a rejection by at least one of the subject, the provider, and the third party regarding participation in the at least one clinical trial conducted by the fourth party.

92. The method of claim 90, wherein the notifying is transmitted to the testing kit.

93. The method of claim 90, wherein the notifying is provided to at least one of the subject, the provider, and the third party in conjunction with the transmittal of the test results.

94. The method of claim 89, further comprising notifying a fourth party conducting the clinical trials of the identification by the data service of a potential candidate to participate in the at least one clinical trials.

95. The method of claim 94, wherein the notifying is provided to the fourth party conducting the at least one clinical trials without including the identification information of the subject.

96. The method of claim 94, further comprising billing the fourth party conducting the at least one clinical trial for fees associated with determining and reporting the existence of a potential candidate to participate in the at least one clinical trial.

97. The method of claim 86, wherein the electronic information includes side effects data.

98. The method of claim 97, further comprising the data service presenting the side effects data along with the test results to at least one of the subject, the provider, and the third party.

99. The method of claim 75, further comprising generating at least one of referral and advertising revenue from the generating of the correlation information.

100. The method of claim 75, further comprising storing the correlation information such that the correlation information is accessible to a plurality of third parties.

101. The method of claim 100, wherein access is available to the data service for a fee set by the data service to be paid by the third party.

102. The method of claim 1, further comprising:
when introducing a testing kit to a location proximate the sample, determining a schedule for performing the testing based upon subject history or requirements of the testing kit;
at the data service, determining if the testing has been performed by comparing received electronic information from the testing kit to the required schedule; and
at the data service, transmitting a reminder to at least one of the subject, the provider, and the third party if the testing has not been performed

103. The method of claim 102, further comprising:
issuing one or more reminders to at least one of the subject, the provider, and the third party when said electronic information has not been received according to the schedule; and
issuing a notification to at least one of the subject, the provider, and the third party when said test has not been performed within a preset expiration time.

104. The method of claim 1, further comprising:
the subject being evaluated as a possible candidate for quarantine based upon at least one of potential medical, chemical, and biological condition;
a provider determining a set of desired tests based upon the at least one potential medical, chemical, and biological condition;
preparing a testing kit based on the set of desired tests;
introducing the testing kit to the subject at a quarantine facility;
the testing kit comprising at least one of a disposable sample and a reusable sample collection and transport kit;
collecting at least one sample from the subject in such a method as required by the desired tests;
introducing the at least one sample to the at least one of the disposable and reusable sample collection and transport device;
introducing the at least one disposable or reusable sample collection and transport kit to a reusable testing kit;
the reusable testing kit generating electronic information representative of sample parameters of interest as determined by the reusable testing kit;
at a data service, receiving electronic information generated by the testing kit;
at the data service interpreting the received electronic information to determine if the subject should be quarantined; and
providing a notification to at least one of the provider and the third party of such determination.

105. The method of claim 104, wherein a quarantine facility is part of an immigration facility that screens an immigrating person as the quarantine subject.

106. The method of claim 104, further comprising granting the quarantine subject a clearance based upon the granting notification.

107. The method of claim 106, wherein the clearance is a determination that the quarantine subject is not a substantial health threat outside of the quarantine facility.

108. The method of claim 107, further comprising the step of allowing the quarantined subject to leave the quarantine facility.
